(19) United States
(12) Patent Application Publication
(10) Pub. No.: US 2025/0320255 A1
(43) Pub. Date: Oct. 16, 2025

(54) UNIVERSAL ADJUVANT FOR NASAL, ORAL, AND INTRAMUSCULAR DELIVERY OF VACCINES

(71) Applicant: Design-Zyme LLC, Lawrence, KS (US)

(72) Inventors: Peter Petillo, Lawrence, KS (US); Dwight Deay, III, Lawrence, KS (US); Michael Branden, Lawrence, KS (US); Erik Naylor, Lawrence, KS (US); Mary Beth Carter, Lawrence, KS (US)

(21) Appl. No.: 18/869,181

(22) PCT Filed: May 25, 2023
(86) PCT No.: PCT/US23/23550

§ 371 Date: Nov. 25, 2024

Related U.S. Application Data
(60) Provisional application No. 63/365,432, filed on May 27, 2022.

(51) Int. Cl. C07K 14/005; A61K 39/00

(52) U.S. Cl. CPC C07K 14/005; A61K 2039/55555; A61K 2039/55583; C12N 2740/16034; C12N 2760/16134

(57) ABSTRACT

Self-adjuvanting vaccine compositions comprising at least one modified immunogen via in vitro glycosylation methods that provide a rational approach for generating glycosylated versions of immunogens via the reducing end of a linear carbohydrate, the reducing end containing an N-acyl-2-amino moiety. Self-adjuvanting vaccine compositions comprising a plurality of heterologous immunogens associated with a multivalent carrier, wherein at least one immunogen is glycosylated to allow for mucosal delivery. Self-adjuvanting vaccine compositions comprising multivalent carriers and related methods using the self-adjuvanting vaccine compositions in various therapeutic and prophylactic applications for inducing an immune response against, treating, or preventing a bacterial, viral, fungal, or protozoan infection. Pathogens for which this approach may be useful include, but are not limited to, influenza viruses, rhinoviruses, human immunodeficiency viruses (HIV), respiratory syncytial virus (RSV), coronaviruses, Babesia, Borrelia, Neisseria, and Chlamydia, and the related diseases thereof.

### CROSS-REFERENCE TO RELATED APPLICATIONS

The present application claims the priority benefit of U.S. Provisional Patent Application Ser. No. 63/365,432, filed 27 May 2022 (27.05.2022), entitled A UNIVERSAL ADJUVANT FOR NASAL, ORAL, AND INTRAMUSCULAR DELIVERY OF VACCINES and U.S. patent application Ser. No. 18/196,214, filed 11 May 2023 (11.05.2023), entitled UNIVERSAL ADJUVANT FOR NASAL, ORAL, AND INTRAMUSCULAR DELIVERY OF VACCINES incorporated by reference in their entity herein.

### FIELD OF INVENTION

The present invention relates generally to the field of immune medicine, in particular, to self-adjuvanting vaccines.

### DESCRIPTION OF THE PRIOR ART

Vaccination constitutes one of the most cost-effective preventative measures against illness and death from infection. Conventional vaccine production techniques using whole pathogens as a vaccine candidate have now transitioned to using subunit components such as recombinant proteins, peptides, or polysaccharides derived from the pathogens. However, subunit components such as proteins, peptides, or polysaccharides alone are poorly immunogenic as they are not easily recognized by immune cells as a foreign body. These immunogens tend to have a low permeability and oral absorption due to their high molecular weight and hydrophilic character. In addition, both proteins and peptides are susceptible to enzymatic degradation, conferring short half-lives in vivo. Consequently, subunit vaccines need the assistance of adjuvants and/or delivery systems (Bashiri et al., 2020).

Adjuvants are immunomodulatory compounds that are used with immunogens (antigens) in vaccine formulations to increase, improve, or boost an immune response (Li et al., 2021).

Adjuvants as a vaccine delivery system can protect antigens, provide sustained release of antigens, target antigens to local lymph nodes, and facilitate immune responses against delivered antigens (Zinkernagel et al., 1997). Moreover, immunomodulatory adjuvants stimulate cellular uptake of antigens from administration sites, activate antigen-presenting cells (APCs) such as B-cells and T-cells, and up-regulate cytokines and chemokines to provide a robust adaptive immune response (Fearon, 1997).

Currently, the only FDA-approved adjuvants for use in humans are aluminum salts, AS03, AS04 (Monophosphoryl lipid A (MPL)+aluminum salt), MF59 (oil in water emulsion composed of squalene), AS01_{B} (monophosphoryl lipid A (MPL) and QS-21, combined in a liposomal formulation), and CpG 1018 (cytosine phosphoguanine (CpG), a synthetic form of DNA that mimics bacterial and viral genetic material). These adjuvants are only approved for administration via injection. To date, there are no adjuvants approved for the mucosal delivery of vaccines in the U.S. Mucosal modes of administration include, but are not limited to, oral, intranasal, intravaginal, intrarectal, intraocular, and intravitreal.

Adjuvanted vaccines can cause local reactions, such as redness, swelling, induration, and pain at the injection site, and systemic reactions, such as fever, chills, rashes, and body aches. (Hervé et al., 2019). There is a recognized need in the art for safe adjuvants which can be co-administered as part of a vaccine composition in order to stimulate a response by the immune system to the antigen or antigens that are also part of the vaccine composition. The adjuvant helps the immune system to generate a more robust antibody response to the antigen or antigens than would be seen if the antigen or antigens were injected alone.

Currently almost all vaccines are administered by injection. While injection is effective, the use of needles carried the risks of both infection at the injection site and transmission of infectious diseases, the treatment of which incurs very significant costs. In addition, trained personnel are required to administer vaccines by injection, due to the aforementioned risks. These problems are particularly relevant in low and middle income countries (LMICs). An advantage of mucosal delivery of a vaccine is that it can be delivered other than by way of injection through needles, thereby providing an immunization regime which may be much safer and more suited to mass immunization, and therefore more attractive to mass vaccination in LMICs. This is especially true for the oral delivery of a vaccine via a pill.

More than 90% of all infections use the mucosa as portals of entry. Advantages of mucosal immunization include: local production of secretory immunoglobulin A (sIgA) which blocks epithelial colonization and penetration of pathogens into the body; immunization of one mucosal site often induces immune response in other mucosal effector tissues (Lawson et al., 2011); and the production of mucosal antibodies (IgA) can prevent systemic infection (Gupta et al., 2015, MacPherson et al., 2008).

While complete protection against many infectious agents would, in addition, require the induction of systemic humoral immunity (particularly IgG antibodies) and cytotoxic T lymphocytes (CTLs), generation of sIgA, at the mucosa, especially the nasal mucosa, may also result in reduced disease transmission.

Adjuvants that work for systemic immunization, such as alum, are generally not effective for mucosal immunization. Moreover, traditionally administered vaccines do not promote high/effective levels of mucosal immunity.

Hyaluronic acid (HyA) is a natural polysaccharide with a linear structure of repeating disaccharide units composed of D-glucuronic acid and N-acetyl-D-glucosamine. HyA has a proven clinical safety in humans, as it has been widely used for medical products (Becker et al., 2009). More recently, attempts have been made to use HyA as a vaccine adjuvant, as HyA can act as both an immunostimulatory agent and vaccine delivery system. JPH05163161 discloses a vaccine composition for intranasal inoculation which consists of an influenza vaccine and hyaluronic acid, or salt thereof, wherein the HyA is not covalently attached to the vaccine. KR2015014149 discloses either reductive amination to randomly oxidized glucuronic rings or amidation with the carboxylic acid moiety of a glucuronic acid to form HyA-peptide conjugates for transdermal or transmucosal delivery. The conjugate contains 1 to 10 molecules of peptide per hyaluronic acid. US 2021/0393758 and U.S. Pat. No. 9,034,624 disclose derivatizing a GAG through the carboxylic acid moiety of a glucuronic acid with a linker terminated in an aldehyde moiety. This moiety can then be reductively aminated with an amine moiety of a biologically active molecule, typically the N-terminus of a polypeptide or protein chain. U.S. Pat. No. 6,824,793 discloses that the mucosal delivery of esterified auto-crosslinked HyA polymers, in combination with an antigen of interest, acts to enhance the immunogenicity of the co-administered antigen. The HyA derivatives are provided as microspheres that either adsorb or physically incorporate the antigen and provide the best results when co-administered with an adjuvant. Suzuki et al. disclose HyA-coated micelles containing antigens and adjuvants for nasal delivery. In all cases, these attempts to create a HyA adjuvant have drawbacks which are overcome by the present invention.

### SUMMARY OF THE INVENTION

The present invention is broadly concerned with a vaccine composition comprising at least one modified immunogen, and methods of modifying the immunogen via in vitro glycosylation methods that provide a rational approach for generating glycosylated versions of immunogens having a linear carbohydrate moiety via the reducing end of the carbohydrate, said reducing end containing an N-acyl-2-amino moiety.

### BRIEF DESCRIPTION OF THE DRAWINGS

FIG. 1 is a representative SDS-PAGE of key SEC fractions from a mosaic-8-OspC mi3 nanoparticle assembly reaction.

FIG. 2 is a representative flow cytometry composite of DC selection based on CD11c+ subsets and further refined for MHC-II presentation.

FIG. 3 are representative ELISA immunoassay composites showing area under the curve analysis of serum IgG production after 8 weeks post IM injection of 5 μg of protein/protein+HyA of BALB/c mice with single boost 4 weeks post prime, (left panel) dilution series (100×-6400×), and (right panel) dilution series (100×-102,000×). Error bars represent SEM.

FIG. 4 are representative ELISA immunoassay composites of serum IgG for animals orally dosed (2×week/8 total doses−left panel; 1×week/4 total doses−right panel) with 40 ug HyA50-mosaic-8-RBD-mi3 (n=10) and 40 ug mosaic-8-RBD-mi3 (n=5). Data represent ED50s for dilution series (100×-1648400×) against SARS-2-RBD Beta protein. Error bars represent SEM.

FIG. 5 is a representative ELISA immunoassay composite of serum IgG and nasal sIgA for animals nasally dosed (1×week/4 total doses) with 10 ug HyA50-mosaic-8-RBD-mi3 (n=10) and 10 ug mosaic-8-RBD-mi3 (n=5). Data represent area under the curve for dilution series IgG (500×-12500×) and IgA (100×-2500×) against SARS-2-RBD Beta protein. Error bars represent SEM.

FIG. 6 is a representative ELISA immunoassay composite of serum IgG for animals nasally dosed IM (prime and boost) with 10 ug HyA50-mosaic-8-OspC-mi3 (n=5) and 10 ug mosaic-8-OspC-mi3 (n=5). Data represent area under the curve for dilution series IgG (100×-1648400×) OspC protein. Error bars represent SEM. Individual animals are shown as circles, etc.

FIG. 7 is a pictorial representation of the HyA covalently attached to the (left panel) mosaic-8-RBD mi3 nanoparticle and, (right panel) mosaic-8-OspC mi3 nanoparticle.

### DETAILED DESCRIPTION OF THE INVENTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank and other databases referred to herein, are incorporated by reference in their entirety with respect to the related technology.

Adjuvants are immunomodulatory compounds that are used with immunogens (antigens) in vaccine formulations to increase, improve, or boost an immune response. One skilled in the art would recognize that while traditional vaccines are formulated into mixtures of an immunogen (antigen) plus an adjuvant, vaccines in which the two moieties are contained within a single molecule are designated as self-adjuvanting vaccines. Thus, in the present invention, “self-adjuvanting” refers to an adjuvant that is covalently attached to the immunogen. A covalently attached adjuvant cannot diffuse away from the immunogen, and is sufficient to increase, improve, or boost an immune response, in the absence of traditional, non-covalently attached adjuvants.

The present invention provides a vaccine composition, comprising at least one modified immunogen. The method of modifying the immunogen comprises in vitro glycosylation methods that provide a rational approach for generating glycosylated versions of immunogens via the reducing end of a linear carbohydrate. This invention allows for the custom control of a linear carbohydrate placed on the immunogen including those that may be stabilizing. The glycosylated immunogen of the vaccine composition may be self-adjuvanting, for example, for enteral, parenteral, topical, intravaginal, intranasal, intrarectal, intraocular, and/or intravitreal delivery.

As used herein, the terms “antigen” or “immunogen” are used interchangeably to refer to a substance, typically a peptide or a protein, which is capable of inducing an immune response in a subject (e.g., a mammal, such as a human). The term also refers to peptides and proteins and derivatives thereof that are immunologically active in the sense that once administered to a subject, are capable of or intended to evoke an immune response of the humoral and/or cellular type directed against that protein or peptide or a variant thereof. The immunogens of the current invention can be recombinant or synthetic. An immunogen for use in a vaccine can be derived from a whole inactivated organism, a protein, a protein fragment, a subunit protein, or any combination thereof that are part of, or derived from the pathogen, and that will induce the immune response. Immunogens for use in vaccines can be identified by a variety of methods known to one of skill in the art (e.g., Earnhart et al., 2007; Olsen et al., 2015, He et al., 2021).

In one embodiment of this invention, the vaccine composition comprises at least one immunogen having at least one amino nucleophilic moiety. See FIG. 7. The at least one amino nucleophilic moiety is covalently glycosylated through the reducing end of a linear carbohydrate, wherein the linear carbohydrate is functionalized at the reducing end with an oxazoline. The glycosylated immunogen has a molecular weight of at least about 7,500 Daltons.

The linear carbohydrate is functionalized at the reducing end with an oxazoline. The at least one amine moiety of the immunogen covalently binds to the oxazoline of the linear carbohydrate to form one or more amidine moieties (Scheme 1 and Scheme 2), utilizing the methodology as described in U.S. Pat. Nos. 11,021,730 and 11,643,376. Scheme 3 shows schematically how the amidine forms at the reducing end of a hyaluronic acid polymer after covalent conjugation to a protein, including protein immunogens of the present invention. These tautomers may be in equilibrium with each other, and other structures related to these two canonical structures.

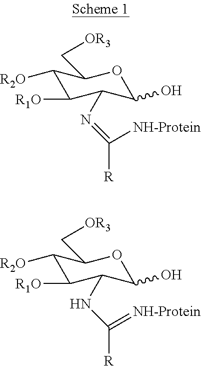

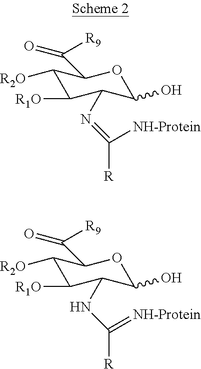

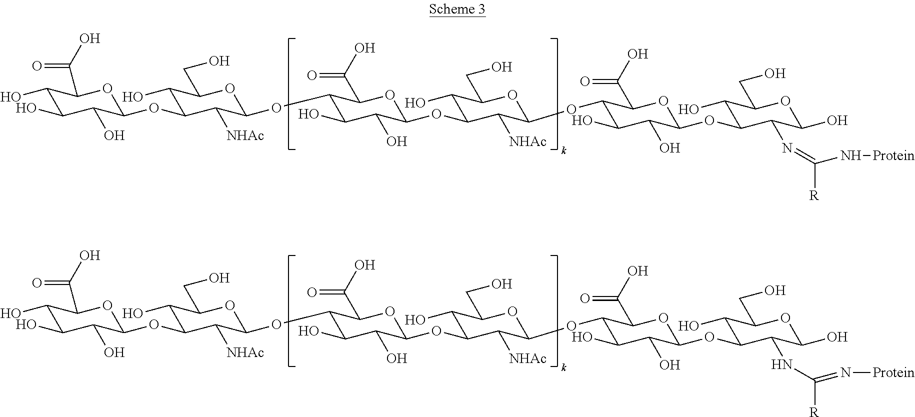

There is no stereochemical requirement at C2, C3, C4, C5 and C6 of the carbohydrate, and the N-acyl moiety may be either equatorially or axially disposed and still result in formation of an oxazoline. One of ordinary skill in the art will recognize that the C3, C4 and C6 hydroxyl moieties on the parent N-acyl-2-amino carbohydrate may be further substituted with other carbohydrates to form larger linear oligosaccharides.

Each R is individually and independently selected from the group consisting of C₁-C₆ alkyls, branched C₃-C₈ alkyls, (CH₂)ₘ—CN, (CH₂)ₘOR₆, (CH₂)ₘ—CO₂H, (CH₂)ₘ—CO₂R₆, (CH₂)ₘ—NR₆(R₇), (CH₂)ₘ—S(O)ₙ—C1-C6alkyl, (CH₂)ₘ—C(O)NR₆(R₇), (CH₂)ₘ—CO₂—C4-C6 heterocyclyl, (CH₂)ₘ-C4-C6 heterocyclyl, (CH₂)ₘ—CO₂—C4-C6 heteroaryl, and (CH₂)ₘ—C₄-C₆-heteroaryl, wherein each alkyl may optionally contain an ether linkage and, wherein each alkyl is optionally substituted with one or two C₁-C₆ alkyls;
R₁, R₂ and R₃ are each independently selected from the group consisting of H, saccharides including but not limited to glucosamines, acetylated glucosamines, uronic acids, and polymeric carbohydrates taken from the group consisting of chitin, partially deacylated chitin, chitosan, partially acylated chitosan, hyaluronic acid, keratin, keratin sulfate, chondroitin, chondroitin sulfate, dermatan, dermatan sulfate, heparin, and derivatives thereof;
   each R₆ and R₇ is individually and independently H, C1-C6 alkyl, or branched C3-C8 alkyl;
   each k is individually and independently 0-1,000;
   each m is individually and independently 1, 2, 3, 4, or 5;
   each n is individually and independently 0, 1, or 2;
   each R₈ is individually and independently H, C1-C6 alkyl, or branched C3-C8 alkyl, wherein
   each alkyl may optionally contain an ether linkage and, wherein each alkyl is optionally substituted with an aryl group or one or two C₁-C₆ alkyl;
R₉ is selected from the group consisting of OR₈, NHR₈, and NR₈R₈.

Suitable linear carbohydrates include, but are not limited to, chitin, partially deacylated chitin, chitosan, partially acylated chitosan, hyaluronic acid, keratin, keratin sulfate, chondroitin, chondroitin sulfate, dermatan, dermatan sulfate, and heparin, and derivatives thereof. In one embodiment, the linear carbohydrate has a molecular weight of at least about 500 Daltons, at least about 1,000 Daltons, at least about 1,500 Daltons, at least about 3,500 Daltons, at least about 5,000 Daltons, at least about 10,000 Daltons, at least about 20,000 Daltons, at least about 25,000 Daltons, at least about 33,000 Daltons, at least about 50,000 Daltons, and at least about 120,000 Daltons.

In another embodiment, the linear carbohydrate has a molecular weight of less than about 50,000 Daltons, or less than about 6,000 Daltons. In another embodiment, the linear carbohydrate has a molecular weight of about 10,000 Daltons to about 120,000 Daltons, about 20,000 Daltons to about 80,000 Daltons, and about 30,000 Daltons to about 50,000 Daltons. In a preferred embodiment, the linear carbohydrate has a molecular weight of about 110,000 Daltons, about 50,000 Daltons, about 40,000 Daltons, about 30,000 Daltons, and about 20,000 Daltons.

The linear carbohydrate according to the present invention is not limited by its source or origin and encompasses those obtained from natural origins including those from human and other mammalian sources, those produced by genetically engineered animal cells, plant cells, microorganisms, and other cells, those enzymatically manufactured, those manufactured by fermentation processes, those artificially synthesized by chemical processes and others. The linear carbohydrate may encompass monosaccharides, disaccharides, oligosaccharides, polysaccharides, and modified derivatives thereof, so long as the reducing end saccharide moiety is unprotected on C1 (i.e., contains a hemiacetalic hydroxyl) and contains an N-acyl-2-amino moiety.

In another embodiment, the linear carbohydrate is hyaluronic acid (HyA), preferably with a molecular weight of between about 6 and about 75 kD. In a more preferred embodiment, the size of the hyaluronic acid is between about 20 and about 50 kD. In an embodiment, the size of the hyaluronic acid is about 110,000 Daltons, about 50,000 Daltons, about 40,000 Daltons, about 30,000 Daltons, and about 20,000 Daltons. In a more preferred embodiment, the size of the hyaluronic acid is about 50,000 Daltons.

In one embodiment, the linear carbohydrate when covalently attached to the immunogen is self-adjuvanting. In a preferred embodiment, hyaluronic acid as an adjuvant is similar or superior to already known adjuvants, for example: Aluminum (amorphous aluminum hydroxyphosphate sulfate (AAHS), Alhydrogel®, aluminum hydroxide, aluminum phosphate, Alum), Quil-A®, Addavax™, Complete Freund's Adjuvant (CFA), AS03, AS04, MF59®, AS01_{B}, MPL®, CpG 1018, Poly I:C, Poly I:C12U, Poly I:CLC, Flagellin-based, Resiquimod-based (i.e., R848), Glucopyranosyl Lipid Adjuvant (GLA), Chitosan, LPS, Matrix-M™, Montanide ISA™51 (incomplete Freud's adjuvant), and Montanide ISA™720.

The term “modified carbohydrate” (or “modified derivative thereof”) used herein may refer to those modified through any process of isolation, separation and purification from naturally-occurring sources and origins, those that have been enzymatically modified, those that have been chemically modified, those that have been modified by biochemical means, including microorganisms, wherein such modifications may comprise those known in the field of glycoscience, for example, alkylation, hydrolysis, oxidation, reduction, esterification, acylation, amidation, amination, etherification, nitration, dehydration, glycosylation, phosphorylation, and sulfation. One skilled in the art would recognize the moieties, including but not limited to hydroxyl, carboxylate, and amide, within the linear carbohydrate could be modified by these methods. In an embodiment, the modification of the carboxyl group is at least about 25%. In another embodiment, the modification of the carboxyl group is at least about at least about 50%, In a preferred embodiment, the modification of the carboxyl group is at least about 75%. In one embodiment, the carboxyl groups are esterified with an alkyl group, i.e., methyl, ethyl, propyl, dodecyl, and pentyl benzyl. In another embodiment, carboxyl groups which are not esterified with an alkyl group as above, may be reacted with lipid chain/alkyl residues from a C₁₀₋₂₀ aliphatic alcohol to produce “mixed” esters. The modified or unmodified carbohydrate is not cross-linked to hydroxyl groups of the same or different modified or unmodified carbohydrate molecule.

The at least one immunogen may already be N-glycosylated or O-glycosylated as known to one skilled in the art, before applying the glycosylation methods of the invention, thereby providing a hyper-glycosylated immunogen. An immunogen may be post-transitionally modified either by natural or synthetic means. Post-translational modification (PTM) is the chemical modification of a protein after its translation and involves the later steps in protein biosynthesis for many proteins.

One or more PTMs may occur on the same immunogen. PTMs can occur in vivo (natural) or in vitro (synthetic). A list of natural PTMs includes, but is not limited to, ADP-ribosylation, glycosylation, glypiation, isoprenylation, methylation, myristoylation, oxidation, sulfation, palmitoylation, phosphorylation, prenylation, and polysialylation. A list of synthetic PTMs includes, but is not limited to, amidation, biotinylation, glycation, methylation, oxidation, pegylation, phosphorylation, reductive amination, and sulfation.

The location of the at least one amino nucleophilic moiety is independently and individually selected from the group consisting of the C terminus, the N terminus, and internally of the immunogen. In one embodiment, the amino nucleophilic moiety of the immunogen is independently and individually selected from the group consisting of lysine, arginine, histidine, and the N-terminal amine moiety of the immunogen. In a preferred embodiment, the amino nucleophilic moiety of the immunogen is lysine. In another preferred embodiment, the amino nucleophilic moiety is the N-terminal amine moiety of the immunogen. The amine nucleophilic moiety may be natural or unnatural. That is, the amine nucleophilic moiety may be present in the native immunogen, or the native immunogen may be “modified” (or “modified derivative thereof”) indicating those modified through any process of isolation, separation, and purification from naturally occurring sources and origins, those that have been enzymatically modified, those that have been chemically modified, and those that have been modified by biochemical means, including microorganisms.

A pathogen is an agent that causes disease or illness in a host (i.e., human), including but not limited to, bacteria, fungi, viruses, helminths, and protozoa.

In an embodiment, the immunogens of the instant application are derived from pathogens. In another embodiment, the immunogens of the instant application are derived from pathogens, selected from, but not limited to, protozoa, fungi, helminths, bacteria, and viruses. In preferred embodiment, the immunogen is derived from a viral or bacterial pathogen. In a more preferred embodiment, the immunogen is derived from a viral pathogen. Immunogens for use in vaccines can be identified by a variety of methods known to one of skill in the art.

In another embodiment, the protozoan pathogens are selected from the group comprising, but not limited to, the genera Acanthamoeba, Babesia, Entamoeba, Giardia, Naegleria, Plasmodia, Toxoplasma, Trichomonas, and Trypanosoma.

In another embodiment, the helminthic pathogens are selected from the group comprising, but not limited to, the genera Ancylostoma, Ascaris, Clonorchis, Diphyllobothrium, Echinococcus, Echinocococcus, Enterobius, Fasciola, Necator, Onchocerca, Schistosoma, Strongyloides, Taenia, Trichuris, and Wuchereria, In another embodiment, the fungal pathogens are selected from the group comprising, but not limited to, the genera Arthrodermataceae, Aspergillus, Basidiobolus, Blastomyces, Candida, Coccidioides, Conidiobolus, Cryptococcus, Epidermophyton, Eumycetoma, Fusarium, Histoplasma, Lacazia, Malassezia, Microsporum, Mucormycetes, Paracoccidioides, Pneumocystis, Pseudoallescheria, Rhizopus, Sporothrix, Stachybotrys, Talaromyces, Trichophyton, and Trichosporon.

In another embodiment, the bacterial pathogens are selected from the group comprising, but not limited to, the genera Achromobacter, Acanthocheilonema, Acinetobacter, Aeromonas, Anaplasma, Bacillus, Bacteroides, Bartonella, Borrelia, Bordetella, Brucella, Burkholderia, Campylobacter, Capnocytophaga, Chlamydia, Citrobacter Clostridium, Corynebacterium, Coxiella, Ehrlichia, Eikenella, Enterococcus, Escherichia, Francisella, Fusobacterium, Gardnerella, Haemophilus, Helicobacter, Klebsiella, Lactobacillus, Legionella, Leptospira, Listeria, Moraxella, Morganella, Mycobacterium, Mycoplasma, Neisseria, Nocardia, Pasteurella, Peptostreptococcus, Porphyromonas, Propionibacterium, Proteus, Providencia, Pseudomonas, Rickettsia, Salmonella, Schistosoma, Serratia, Shigella, Staphylococcus, Streptococcus, Treponema, Ureaplasma, Vibrio, and Yersinia.

In another embodiment, the viral pathogens are selected from the group comprising, but not limited to, the genera Alphacoronavirus, Alphapapillomavirus, Alphatorquevirus, Alphavirus, Arenavirus, Bornavirus, Betacoronavirus, Betapapillomavirus, Cardiovirus, Coltvirus, Cosavirus, Cytomegalovirus, Deltaretrovirus, Deltavirus, Dependovirus, Dependoparvovirus, Ebolavirus, Enterovirus, Erythrovirus, Flavivirus, Gammapapillomavirus, Hantavirus, Henipavirus, Hepacivirus, Hepatovirus, Hepevirus, Influenzavirus A, Influenzavirus B, Influenzavirus C, Jeilongvirus, Kobuvirus, Lentivirus, including human immunodeficiency virus, Lymphocryptovirus, Lyssavirus, Mamastrovirus, Marburgvirus, Mastadenovirus, Molluscipoxvirus, Morbillivirus, Mupapillomavirus, Nairovirus, Norovirus, Nupapillomavirus, Orthobunyavirus, Orthohepadnavirus, Orthohepevirus, Orthopneumovirus, Orthopoxvirus, Parapoxvirus, Parechovirus, Pegivirus, Phlebovirus, Picobirnavirus, Polyomavirus, Posavirus, Respirovirus, Rhadinovirus, Rhinovirus, Rosavirus, Roseolovirus, Rotavirus, Rubivirus, Rubulavirus, Salivirus, Sapovirus, Seadornavirus, Simplexvirus, Spumavirus, Thogotovirus, Torovirus, Varicellovirus, and Vesiculovirus.

In an embodiment the immunogens are derived from pathogens selected independently and individually from the group comprising Influenzavirus A, Influenzavirus B, Influenzavirus C, Rhinovirus, Lentivirus, including human immunodeficiency virus, Respirovirus including respiratory syncytial virus (RSV), Orthopneumovirus including human Orthopneumovirus, Alphacoronavirus, Betacoronavirus including Middle East respiratory syndrome-related coronavirus (MERS-CoV), SARS-CoV, and COVID-19, Flavivirus including dengue viruses, Zika virus, and West Nile virus, Hepatovirus including hepatitis A, B, C, D, and E, Norovirus, Marburgvirus including Marburg virus, Orthopoxvirus, Togaviridae, Ebolavirus including Ebola virus, Borrelia, Staphylococcus including methicillin-resistant Staphylococcus aureus (MRSA), Legionella, Chlamydia, Plasmodia, Streptococcus pneumoniae, Vibrio cholerae, Listeria, Clostridia, Salmonella, Bordetella, Babesia, Enterococci, Treponemia, Amoeba, Neisseria, and Giardia.

It is understood by one skilled in the art that for each pathogen listed, one or more serotypes may be relevant to a particular disease state caused by the pathogen, including serotypes not yet identified. In an embodiment, the immunogens are derived from Betacoronavirus, more specifically SARS-related emergent zoonotic coronaviruses. In a preferred embodiment, the immunogens are derived from SARS-CoV, MERS-CoV, and SARS-CoV-2 (COVID-19). In some embodiments, the vaccine composition comprises one or more immunogens from the same pathogen genus. That is, the vaccine composition comprises two, three, four, or more different from one another immunogens from the same pathogen genus.

In one embodiment, the glycosylated immunogen described herein can induce the production of detectable antigen-specific antibodies individually and independently selected from the group consisting of IgG, IgA, sIgA, IgD, IgE, IgM, neutralizing antibodies, and cross-reactive neutralizing antibodies thereof.

In another embodiment, the glycosylated immunogen described herein can induce non-specific immune responses (e.g., antibody-dependent cellular cytotoxicity (ADCC), antibody-dependent cellular phagocytosis (ADCP), and antibody-mediated complement-dependent cytotoxicity (CDC)) in response to an antigen. ADCC is when viral antigens on the surface of an infected cell are recognized by specific antibodies. Said antibodies signal natural killer cells to destroy the infected cell via secreted compounds (e.g., cytotoxic granules and cytokines). In ADCP, an infected cell is recognized by specific antibodies which then signal the cell to be destroyed via macrophage-directed phagocytosis. In CDC, antibodies recruit and activate components of the complement cascade, leading to the formation of a Membrane Attack Complex on the cell surface and subsequent cell lysis.

In some embodiments, the immunogens are associated with a carrier. In one embodiment, a vaccine composition, comprising a plurality of immunogen molecules associated with a carrier wherein at least one immunogen of the plurality of immunogen molecules has at least one amino nucleophilic moiety wherein the at least one amino nucleophilic moiety is covalently glycosylated through the reducing end of a linear carbohydrate functionalized with an oxazoline. See FIG. 7.

The glycosylated immunogen has a molecular weight of at least about 7,500 Daltons. The linear carbohydrates and immunogens are as described above herein.

In another embodiment, one or more linear carbohydrates of the present invention are covalently attached to the immunogens of the plurality of immunogens associated with a carrier. In another embodiment, one, two, three, four, or more linear carbohydrates of the present invention are covalently attached to one or more immunogens associated with a carrier. In another embodiment, at least one immunogen of the plurality of immunogens associated with a carrier is not glycosylated. In an example, only one linear carbohydrate is covalently attached to only one immunogen of the plurality of immunogens associated with a carrier. In another example, two linear carbohydrates of the present invention may be attached to one immunogen associated with a carrier or two linear carbohydrates of the present invention may be attached to two different immunogens associated with the same carrier.

A carrier, as used herein, can be generally referred to as a biocompatible molecular system having the capability of incorporating and transporting molecules (e.g., therapeutic agents such as immunogens, glycosylated immunogens, and derivatives thereof) to enhance their selectivity, bioavailability, and efficiency. One of ordinary skill in the art would also refer to a carrier as a scaffold. The carriers used in the methods, compositions, and systems herein described can be a biocompatible molecular system, either naturally occurring or synthetic, that can be functionalized or conjugated for coupling (e.g., covalently or non-covalently) to a plurality of protein immunogens (antigens) or immunogen polypeptides described herein. The carriers can comprise nanoparticles, nanotubes, nanowires, dendrimers, liposomes, ethosomes and aquasomes, polymersomes and niosomes, foams, hydrogels, cubosomes, quantum dots, exosomes, macrophages, and others identifiable to a person skilled in the art.

In some embodiments, the carrier used herein can be a nanosized carrier such as a nanoparticle. As used herein, the term “nanoparticle” refers to a nanoscopic particle having a size measured in nanometers (nm). Size of the nanoparticles may be characterized by their maximal dimension.

The term “maximal dimension” as used herein can refer to the maximal length of a straight-line segment passing through the center of a nanoparticle and terminating at the periphery. In the case of substantially spherical nanoparticles, the maximal dimension of such nanosphere corresponds to its diameter. The term “mean maximal dimension” can refer to an average or mean maximal dimension of the nanoparticles and may be calculated by dividing the sum of the maximal dimension of each nanoparticle by the total number of nanoparticles. Accordingly, value of maximal dimension may be calculated for nanoparticles of any shape, such as nanoparticles having a regular shape such as a sphere, a hemispherical, a cube, a prism, or a diamond, or an irregular shape. The nanoparticles provided herein need not be spherical and can comprise, for example, a shape such as a cube, cylinder, tube, block, film, and/or sheet. In some embodiments, the maximal dimension of the nanoparticles is in the range from about 1 nm to about 5000 nm, such as between about 20 nm to about 1000 nm, about 20 nm to about 500 nm, about 20 nm to about 300 nm, about 20 nm to about 100 nm, or about 20 to about 50 nm.

The nanoparticle can be, but is not limited to, any one of lipid-based nanoparticles (nanoparticles where the majority of the material that makes up their structure are lipids, e.g., liposomes or lipid vesicles), polymeric nanoparticles, inorganic nanoparticles (e.g., magnetic, ceramic, and metallic nanoparticles), surfactant-based emulsions, silica nanoparticles, virus-like particles (particles primarily made up of viral structural proteins that are not infectious or have low infectivity), peptide or protein-based particles (particles where the majority of the material that makes up their structure are peptides or proteins) and/or nanoparticles that are developed using a combination of nanomaterials such as lipid-polymer hybrid nanoparticles formed by polymer cores and lipid shells or nano lipoprotein particles formed by a membrane forming lipid arranged in a membrane lipid bilayer stabilized by a scaffold protein as will be understood by a person skilled in the art.

In some embodiments, a nanoparticle carrier is made up of a plurality of monomeric subunits which assemble with one another through covalent and/or non-covalent forces to form the carrier.

In some embodiments, the carrier described herein is a protein nanoparticle comprising a plurality of particle-forming proteins, which are the monomeric subunit proteins that form the protein nanoparticle. Protein nanoparticles can be categorized into non-viral protein nanoparticles and viral-like particles (VLPs). Examples of non-viral protein nanoparticles include, but are not limited to, ferritins, vaults, heat-shock proteins, chaperonins, lumazine synthase, encapsulins, and bacterial microcompartments. VLPs can be derived from viruses including, but not limited to, adenovirus, cowpea mosaic virus, cowpea chlorotic mottle virus, brome mosaic virus, broad bean mottle virus, bacteriophage lambda (e.g., bacteriophage lambda procapsid), MS2 bacteriophage, Qβ bacteriophage, P22 phage capsid, and others identifiable to a person skilled in the art.

In some embodiments, the nanoparticles described herein comprise a VLP. VLP refers to a non-replicating, viral shell, derived from any of several viruses. VLPs can be naturally occurring or synthesized through the individual expression of viral structural proteins, which can then self-assemble into the virus-like structure. VLPs are generally composed of one or more viral proteins, such as particle-forming proteins referred to as capsid, coat, shell, surface and/or envelope proteins, or particle-forming polypeptides derived from these proteins. In some embodiments, VLPs can form spontaneously upon recombinant expression of the protein in an appropriate expression system. VLPs can differ in morphology, size, and number of subunits. Methods for producing a number of VLPs are known in the art. The presence of VLPs following recombinant expression of viral proteins can be detected using conventional techniques also known in the art, such as by electron microscopy, biophysical characterization, and the like (see e.g., Baker et al., 1991 and Hagensee et al., 1994). For example, VLPs can be isolated by density gradient centrifugation and/or identified by characteristic density banding. Alternatively, cryoelectron microscopy can be performed on vitrified aqueous samples of the VLP preparation in question, and images recorded under appropriate exposure conditions. Any of a variety of VLPs known in the art can be used herein, including but not limited to, Aquifex aeolicus lumazine synthase, Thermotoga maritima encapsulin, Myxococcusanthus encapsulin, bacteriophage Qbeta virus particle, Flock House Virus (FHV) particle, ORSAY virus particle, and infectious bursal disease virus (IBDV) particle. In some embodiments, the nanoparticle used herein can be a bacteriophage VLP, such as Ap205 VLP. In some embodiments, the nanoparticle used herein is a mutated Ap205 VLP (for example, SpyCatcher-CP3, Brune et al., 2016).

In some embodiments, the nanoparticles described herein comprise a self-assembling nanoparticle. A self-assembling nanoparticle typically refers to a ball-shape protein shell with a diameter of tens of nanometers and well-defined surface geometry that is formed by identical copies of a non-viral protein capable of automatically assembling into a nanoparticle with a similar appearance to VLPs. Examples of self-assembling nanoparticle particle-forming proteins include, but are not limited, to ferritin (FR) (e.g., Helicobacter pylori ferritin, see Zhang et al., 2020; Kang et al., 2021), which is conserved across species and forms a 24-mer, as well as B. stearothermophilus dihydrolipoyl acyltransferase (E2p, see He et al., 2021), Aquifex aeolicus lumazine synthase (LuS, see Zhang et al., 2020), and Thermotoga maritima encapsulin (see Hsia et al., 2016; Bruun et al., 2018), which all form 60-mers.

In some embodiments, the self-assembling nanoparticles comprise a plurality of particle-forming proteins of 2-keto-3-deoxy-phosphogluconate (KDPG) aldolase from the Entner-Doudoroff pathway of the hyperthermophilic bacterium Theremotoga Maritima or a variant thereof. In some embodiments, mutations are introduced to the KDPG aldolase for improved particle yields, stability, and uniformity. For example, in some embodiments, mutations can be introduced to alter the interface between the wild-type protein trimer of KDPG aldolase. In some embodiments, the nanoparticle used herein is an i301 nanoparticle or a variant thereof (i.e., Hsia et al., 2016). In some embodiments, the nanoparticle used herein is a mutated i301 nanoparticle (for example, mi3 nanoparticle, Bruun et al., 2018). The self-assembling nanoparticles can form spontaneously upon recombinant expression of the protein in an appropriate expression system. Methods for nanoparticle production, detection, and characterization can be conducted using the same techniques developed for virus-like particles (VLPs).

In some embodiments, the nanoparticles described within comprise an engineered protein complex. An engineered protein complex utilizes rational or computational design to assemble dimeric, trimeric, tetrameric, or pentameric proteins (building blocks) into larger, highly oligomeric complexes. The geometric symmetry and shape of the nanoparticle is determined by the type of building blocks used (for examples see Nguyen et al., 2021). An example of an engineered protein complex includes but is not limited to I53-50 (see Kang et al., 2021).

It is understood that in any of the embodiments disclosed herein, the immunogenic complexes comprise at least one linear carbohydrate covalently attached to at least one immunogen in the plurality of immunogens that may be presented by the nanoparticle carrier. See FIG. 7.

In some embodiments herein described, the nanoparticle carriers used are multivalent carriers. Multivalent carriers can also be referred to as mosaic carriers. As opposed to a monovalent carrier (also referred to as a “homotypic carrier”) which presents a single species of an immunogen, a multivalent nanoparticle carrier presents a heterologous population of immunogens, comprising at least two different immunogens of or derived from different species, or strains selected from the group consisting of, but not limited to, protozoan derived immunogens, fungal derived immunogens, helminthic derived immunogens, bacterial derived immunogens, and viral derived immunogens. In preferred embodiment, the immunogens are viral and/or bacterial derived immunogens. In a more preferred embodiment, the immunogens are viral derived immunogens. In another more preferred embodiment, the immunogens are bacterial derived immunogens. Immunogens are derived from pathogens as described above.

The term “heterologous immunogens” means that the immunogens are of different origins, such as derived from pathogens of different taxonomic groups such as different strains, species, subgenera, genera, subfamilies or families and/or from antigenically divergent pathogens (e.g., variants thereof). Heterogeneous immunogens may be from the same or different genera.

Accordingly, heterologous immunogens presented on a multivalent carrier herein described have different protein sequences.

The heterologous immunogens presented on the multivalent carrier herein described can be displayed on its surface. Alternatively, the heterologous immunogens presented on the multivalent carrier herein described can be partially encapsulated or embedded such that at least an immunogenic portion of the immunogen is exposed and accessible by a host cell receptor so as to induce an immune response. The immunogens presented on the monovalent carrier herein described can be displayed on its surface. Alternatively, the immunogens presented on the monovalent carrier herein described can be partially encapsulated or embedded such that at least an immunogenic portion of the immunogen is exposed and accessible by a host cell receptor so as to induce an immune response.

The term “present” as used herein with reference to a compound (e.g., an immunogen) or functional group indicates attachment performed to maintain the chemical reactivity of the compound or functional group attached. Accordingly, a functional group presented on a nanoparticle carrier is able to perform under the appropriate conditions the one or more chemical reactions that chemically characterize the functional group. A compound presented on a nanoparticle carrier is able to perform under the appropriate conditions the one or more chemical reactions that chemically characterize the compound. For example, where the compound is, or comprises, an immunogen, the immunogen presented by a nanoparticle carrier maintains the complex of reactions that are associated with the immunological activity characterizing the immunogen. Accordingly, presentation of an immunogen indicates an attachment such that the immunological activity associated to the immunogen attached is maintained.

In the instant application, one or more linear carbohydrates are covalently attached to at least one immunogen of the plurality of immunogens covalently attached to the particle-forming proteins of the nanoparticle carrier (e.g., particle-forming proteins of the monovalent or multivalent carrier).

In a preferred embodiment, one or more linear carbohydrates are covalently attached to at least one immunogen of the plurality of immunogens that are covalently attached to the particle-forming proteins of the carrier (e.g., particle-forming proteins of the monovalent or multivalent carrier) through a SpyTag/SpyCatcher binding pair. The SpyTag/SpyCatcher binding pair refers to a protein ligation system that is based on the internal isopeptide bond of the CnaB2 domain of FbaB from Streptococcus pyogenes (see, e.g., Zakeri et al., 2012, Keeble et al., 2019). CnaB2 is split and engineered into two complementary fragments, such that the first fragment (SpyCatcher) is able to bind and form a covalent isopeptide bond with the second fragment (SpyTag) through the side chains of a lysine in SpyCatcher and an aspartate in SpyTag.

In some embodiments, the particle-forming protein of the multivalent carrier is a fusion protein containing amino acid sequences from at least two unrelated proteins that have been joined together, genetically, to express as a single protein. For example, the SpyTag motif can be independently and individually fused to the immunogenic protein. In this same example, the nanoparticle carrier subunit sequence can be fused to a SpyCatcher motif. Alternatively, the SpyCatcher motif can be independently and individually fused to the immunogenic protein. In this same example, the nanoparticle carrier subunit sequence can be fused to a SpyTag motif. Methods to fuse the SpyTag/SpyCatcher motif to the immunogenic protein are known to one of skill in the art as exemplified by Cohen et al., 2021a, Cohen et al., 2022, and Keeble et al., 2019. In one embodiment, the SpyTag motif can be fused C-terminal to the immunogenic protein. In another embodiment, the SpyTag motif can be fused N-terminal to the immunogenic protein. One skilled in the art would recognize that the same sequence relationships between the immunogenic protein relative to the SpyTag motif can occur not only at the termini but as part of an internal sequence of a larger construct. In one embodiment, the SpyCatcher motif can be fused C-terminal to the nanoparticle carrier subunit sequence. In another embodiment, the SpyCatcher motif can be fused N-terminal to the nanoparticle carrier subunit sequence. One skilled in the art would recognize that the same sequence relationships between the nanoparticle carrier subunit sequence and the SpyCatcher motif can occur not only at the termini but as part of an internal sequence of a larger construct.

In one embodiment, the SpyCatcher motif can be fused C-terminal to the immunogenic protein. In another embodiment, the SpyCatcher motif can be fused N-terminal to the immunogenic protein. One skilled in the art would recognize that the same sequence relationships between the immunogenic protein relative to the SpyCatcher motif can occur not only at the termini but as part of an internal sequence of a larger construct. In one embodiment, the SpyTag motif can be fused C-terminal to the nanoparticle carrier subunit sequence. In another embodiment, the SpyTag motif can be fused N-terminal to the nanoparticle carrier subunit sequence. One skilled in the art would recognize that the same sequence relationships between the nanoparticle carrier subunit sequence and the SpyTag motif can occur not only at the termini but as part of an internal sequence of a larger construct.

In some embodiments, the particle-forming protein can be a fusion protein containing a mi3 monomeric subunit protein at the C-terminal of the particle-forming protein and a SpyCatcher protein at the N-terminal of the particle-forming protein or a fusion protein containing a AP205-CP3 monomeric subunit protein at the C-terminal of the particle-forming protein and a SpyCatcher protein at the N-terminal of the particle forming protein such that the SpyCatcher proteins are presented or displayed for binding to the SpyTag of the immunogenic protein. In some embodiments, the vaccine composition comprises a plurality of particle-forming proteins.

One or more of the plurality of particle-forming proteins can, for example, comprise a KDPG aldolase or a variant thereof. In some embodiments, the plurality of immunogen molecules is attached to the particle-forming protein of the plurality of particle-forming proteins. For example, the plurality of immunogen molecules is attached to the particle-forming protein of the plurality of particle-forming proteins through a SpyTag/SpyCatcher binding pair. In one embodiment, the immunogen molecules each individually comprise a SpyTag at the C-terminal of the immunogen molecules and the particle-forming protein of the plurality of particle-forming proteins comprises a SpyCatcher at the N-terminal of the particle-forming protein.

It will be appreciated that the nanoparticle carrier can be configured to present immunogens in a number of variations, combinations, or permutations. For example, the plurality of immunogens presented by a nanoparticle carrier can be individually the same or different, the number of any particular type of immunogens presented by a nanoparticle carrier can vary, the total number of immunogens presented by a nanoparticle carrier can vary, and the ratio of any two or more different immunogen molecule s can vary in different embodiments. It is understood that in each of these embodiments, whether or not specifically stated, the immunogenic complex includes at least one linear carbohydrate covalently attached to at least one immunogen of the plurality of immunogens presented by the nanoparticle carrier. See FIG. 7.

The plurality of immunogen molecules may individually be the same or different. For example, the plurality of immunogen molecules may comprise a first immunogen and a second immunogen that is different from the first immunogen. In some embodiments, the plurality of immunogen molecules can comprise three, four, five, six, seven, eight, nine, ten, eleven, twelve or more different immunogens.

In one embodiment, one immunogen is considered different from another immunogen when the two immunogens are derived from antigenically divergent pathogens. The term “antigenically divergent pathogen” refers to a strain of a pathogen that has a tendency to mutate or has developed mutations over time, thus changing the amino acids that are displayed to the immune system. Such mutation over time can also be referred to as “antigenic drift.”

In some embodiments, the plurality of immunogens are individually derived from different subgenera within the same genus. In some embodiments, the plurality of immunogens are individually derived from different species within the same genus. In some embodiments, the plurality of immunogens individually are derived from different strains within the same species. In some embodiments, the plurality of immunogens are individually derived from different mutations, variants, or strains of a particular pathogen.

For example, the plurality of coronavirus immunogens can be of coronaviruses in the genus of Alphacoronavirus and/or Betacoronavirus. In some embodiments, the plurality of coronavirus immunogens are derived from coronaviruses in the genus of Betacoronavirus. In some embodiments, the plurality of coronavirus immunogens are derived from coronaviruses in the subgenus of Sarbecovirus. In some embodiments, the first coronavirus immunogen and the second coronavirus immunogen are derived from the genus of Betacoronavirus, optionally in the subgenus of Sarbecovirus. For example, the plurality of coronavirus immunogens can be derived from coronaviruses selected from the group comprising SARS, SARS-2, WIV1, SHC014, Rf1, RmYN02, pang17, RaTG13, and Rs4081. For example, the first coronavirus immunogen, the second coronavirus immunogen, or both can be derived from the group comprising SARS, SARS-2, WIV1, SHC014, Rf1, RmYN02, pang17, RaTG13, and Rs4081.

The plurality of immunogen molecules individually attached to a multivalent carrier can be derived from the same protein type or corresponding proteins. One of ordinary skill in the art would understand that immunogen molecules of a same protein type may or may not have identical amino acid sequences, but generally share some sequence homology. In some embodiments, proteins of different immunogen taxonomic groups having the same function are considered the same protein type or corresponding proteins. In some embodiments, immunogens of a same protein type have at least 50% sequence identity, for example at least 65%, 70%, 80%, 90%, 95%, 98%, 99%, or more sequence identity. Alternatively, in some embodiments the immunogens can comprise immunogen proteins of different protein types. One of ordinary skill in the art would understand that immunogen proteins of different protein types typically have different functions.

The total number of heterologous immunogens presented by a multivalent carrier can be different in different embodiments. In some embodiments, the multivalent carrier can comprise a total number of immunogens about, at least, at least about, at most, or at most about 2, 3, 4, 5, 6, 7, 8, 9, 10, 11, 12, 13, 14, 15, 16, 17, 18, 19, 20, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, immunogens.

In some embodiments, the number of any two different immunogen mole c ule s can be in a ratio from 1:100 to 100:1. In some embodiments, the ratio can be, be about, be at least, be at least about, be at most, be at most about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of the values.

It should be understood that the total number of immunogen molecules presented by a nanoparticle carrier is limited by the number of particle-forming subunits that make up the nanoparticle, such as the number of particle-forming lipids in lipid-based nanoparticles and the number of particle-forming proteins in protein-based nanoparticles. For example, encapsulin proteins from Thermotoga maritima form nanoparticles having 60-mers. Therefore, encapsulin-based nanoparticles (e.g., mi3 nanoparticle and i301 nanoparticle) can present a maximum of 60 protein immunogen molecules.

The multivalent carrier described herein can induce broadly protective anti-pathogen responses by eliciting broadly neutralizing antibodies and/or cross-reactive neutralizing antibodies. Broadly neutralizing antibodies are antibodies that can neutralize the pathogen from a taxonomic group that is not only the same as but also differs from the taxonomic groups of the pathogen from which the immunogens used to elicit the antibodies are derived. A broadly neutralizing response can also be referred to as a heterologous neutralizing response. Cross-reactive neutralizing antibodies are antibodies which are both active and neutralizing against immunogens that were not specifically used to raise the antibody in question. That is, the antibody has neutralizing activity beyond the protein immunogens used to raise or create the antibody. In some embodiments, the multivalent carriers described herein can elicit broadly neutralizing antibodies that neutralize one or more pathogens from a genus, subgenus, species, and/or strain that differ from the genus, subgenus, species, and/or strain of the pathogen from which the immunogens are derived to produce the multivalent carriers.

In some embodiments, the multivalent carrier comprising heterologous immunogens derived from a plurality of pathogens including at least a first immunogen and at least a second immunogen that can induce heterologous binding and neutralizing responses and/or cross-reactive neutralizing antibodies against not only the first pathogen and the second pathogen, but also against one or more pathogens different from the first pathogen and the second pathogen from which the immunogens were derived (e.g., a third pathogen, a fourth pathogen, etc.). In particular, the multivalent carrier comprising heterologous immunogens derived from a plurality of pathogens not including one or more particular immunogens can induce heterologous binding and neutralizing responses and/or cross-reactive neutralizing antibodies against the one or more particular pathogens.

In some embodiments, the multivalent carrier comprising heterologous immunogens derived from a plurality of pathogens including a first pathogen and a second pathogen can induce about the same or comparable magnitude (e.g., about, at least, at least about 80%, 85%, 90%, 95%, 100%, 105%, 110%, 115%, 120%, 125%, 130%, 140%, 150%, or a number or a range between any two of these values, relative to one another) of immune response against the first immunogen derived from the first pathogen and/or the second immunogen derived from the second pathogen when compared to a monovalent carrier (“homotypic nanoparticle”) comprising a homologous population of a single immunogen derived from the first pathogen or the second immunogen derived from a second pathogen. In other words, co-display of immunogens from derived pathogens of different taxonomic groups does not diminish the immune response against a pathogen relative to homotypic carriers presenting only immunogens from the particular pathogen.

In a non-limiting example, in terms of the magnitude of the immune response against a particular pathogen, it can be advantageous to conduct immunization with a nanoparticle carrier presenting a plurality of immunogens that includes immunogens derived from one pathogen as well as immunogens derived from other taxonomically distinct yet related pathogens versus immunization with a homotypic carrier presenting a single immunogen type.

In some embodiments, the multivalent carrier comprising heterologous immunogens derived from a plurality of pathogens including a first immunogen derived from a first pathogen and a second immunogen derived from a second pathogen can induce an increased magnitude of immune response against the first pathogen and/or the second pathogen when compared to a monovalent carrier comprising a homologous population of a single immunogen derived from the first pathogen or the second pathogen. The magnitude of immune response induced by the multivalent carrier can be about, at least, or at least about 0.2, 0.5, 0.9, 1.1, 1.5, 1.7, 2, 3, 4, 5, 6, 7, 8, 9, 10, fold(s), or a number or a range between any of these values, greater than by the monovalent carrier.

In some embodiments, the magnitude of immune response induced by the multivalent carrier can be increased by about, at least, or at least about 5%, 10%, 20%, 30%, 50%, 75%, 100%, 110%, 120%, 150%, 200%, 250%, 300%, 400%, 500%, 600%, 700%, 800%, 900%, 1000%, or a number or a range between any of these values, as compared to that by the monovalent carrier.

In some embodiments, the multivalent carrier does not present an immunogen derived from a particular pathogen but can still produce broadly neutralizing antibodies against that particular pathogen, for example, at a comparable or even enhanced magnitude as compared to a monovalent carrier presenting an immunogen derived from that particular pathogen. For example, the multivalent carrier comprising heterologous immunogens derived from a plurality of pathogens not including a first immunogen derived from the first pathogen can nevertheless induce about the same or comparable magnitude (e.g., about, at least, at least about 80%, 85%, 90%, 95%, 100%, 105%, 110%, 115%, 120%, 125%, 130%, 140%, 150%, or a number or a range between any of these values, relative to one another) of immune response against the first pathogen when compared to a monovalent carrier comprising a homologous population of a single immunogen derived from the first pathogen.

In some embodiments, the multivalent carrier comprising heterologous immunogens derived from a plurality of pathogens but not including a first immunogen derived from a first pathogen can elicit an enhanced heterologous binding and neutralizing response against the first pathogen when compared to a monovalent carrier comprising a homologous population of a single immunogen derived from a first pathogen and a second immunogen derived from a second pathogen. The first and second pathogens and the corresponding immunogens derived thereof are different from one another. The magnitude of the neutralizing response induced by the multivalent carrier can be about, at least, or at least about 0.2, 0.5, 0.9, 1.1, 1.5, 1.7, 2, 3, 4, 5, 6, 7, 8, 9, 10-fold, or a number or a range between any of these values, greater than the response by the monovalent carrier. In some embodiments, the magnitude of the immune response induced by the multivalent carrier can be increased by about, at least, or at least about 5%, 10%, 20%, 30%, 50%, 75%, 100%, 110%, 120%, 150%, 200%, 250%, 300%, 400%, 500%, 600%, 700%, 800%, 900%, 1000%, or a number or a range between any of these values, as compared to the response by the monovalent carrier.

In some embodiments, the multivalent carrier comprising heterologous immunogens from a plurality of pathogens including a first immunogen derived from a first pathogen and a second immunogen derived from a second pathogen can elicit a substantially enhanced neutralizing response against the first pathogen and/or the second pathogen when compared to an unattached immunogen from the first pathogen or the second pathogen. The magnitude of the neutralizing response induced by the multivalent carrier can be about, at least, or at least about 1, 2, 3, 4, 5, 6, 7, 8, 9, 10, 50, 100, 200, 300, 400, 500, 600, 700, 800, 900, 1,000-fold, or a number or a range between any of these values, greater than the response by the unattached immunogen.

In one embodiment, the plurality of coronavirus immunogens attached to a multivalent carrier can be of a same protein type or corresponding proteins. Coronavirus immunogens of a same protein type may or may not have identical amino acid sequences, but generally share some sequence homology. For example, the coronavirus S proteins of different coronaviruses are of a same protein type or corresponding proteins. As another example, envelope proteins from different coronaviruses are considered the same protein type or corresponding proteins. In some embodiments, proteins of different coronavirus taxonomic groups having the same function are considered the same protein type or corresponding proteins. Another example is the receptor binding domains of the different coronaviruses. In some embodiments, coronavirus immunogens of a same protein type have at least 50% sequence identity, for example at least 65%, 70%, 80%, 90%, 95%, 98%, 99%, or more sequence identity.

In some embodiments, the immunogen used herein can be derived from a coronavirus spike (S) protein or a portion thereof. A S protein is one of four major structural proteins covering the surface of each virion. The S protein, comprising a S1 subunit and a S2 subunit, is a highly glycosylated, type I transmembrane protein capable of binding to a host-cell receptor and mediates viral entry. The S protein comprises a domain referred to as the RBD that mediates the interaction with the host-cell receptor to enter the host cell after one or more RBDs adopts an “up” position to bind the host receptor. It is believed that after binding the receptor, a nearby host protease cleaves the spike, which releases the spike fusion peptide, facilitating virus entry.

Known host receptors for coronaviruses (e.g., Beta-coronaviruses) include angiotensin-converting enzyme 2 (ACE2), dipeptidyl peptidase-4 (DPP4) or sialic acids. In some embodiments the immunogen is derived from a coronavirus S protein or a portion thereof, comprising or consisting of an amino acid sequence having, having about, having at least, or having at least about, 80%, 85%, 90%, 95%, 98%, 99%, or more sequence identity to the amino acid sequence of any of the coronavirus S proteins from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31.

In some embodiments, the immunogen used herein can be derived from a coronavirus spike RBD or a portion or a variant thereof. The coronavirus spike RBD or a portion thereof used herein can be of any species or strains in the genus of alphacoronavirus and/or betacoronavirus. For example, the coronavirus spike RBD protein or a portion thereof can be of any species or strains in the subgenus of Embecovirus, including but not limited to, Betacoronavirus 1 (e.g., Bovine coronavirus and human coronavirus OC43), China rattus coronavirus HKU24, Human coronavirus HKU1, Murine coronavirus (e.g., mouse hepatitis virus), and Myodes coronavirus 2JL14. The coronavirus spike RBD protein or a portion thereof can be of any viral species or strains in the subgenus of Sarbecovirus, including but not limited to, SARS-CoV, SARS-CoV2, 16B0133, Bat SARS CoV Rf1, Bat coronavirus HKU3 (BtCoV HKU3), LYRa11, Bat SARS-CoV/Rp3, Bat SL-CoV YNLF_31C, Bat SL-CoV YNLF_34C, SHCO14-CoV, WIV1, WIV16, Civet SARS-CoV, Rc-o319, SL-ZXC21, SL-ZC45, Pangolin SARSr-COV-GX, Pangolin SARSr-COV-GD, RshSTT182, RshSTT200, RacCS203, RmYN02, RpYN06, RaTG13, Bat CoV BtKY72, and Bat CoV BM48-31. The coronavirus spike RBD protein or a portion thereof can be of any viral species or strains in the subgenus of Merbecovirus, including but not limited to, Hedgehog coronavirus 1, MERS-CoV, Pipistrellus bat coronavirus HKU5, and Tylonycteris bat coronavirus HKU4. The coronavirus spike RBD protein or a portion thereof can be of any viral species or strains in the subgenus of Nobecovirus, including but not limited to, Eidolon bat coronavirus C704, Rousettus bat coronavirus GCCDC1, and Rousettus bat coronavirus HKU9. The coronavirus spike RBD protein or a portion thereof can be of any viral species or strains in the subgenus of Hibecovirus, including but not limited to, Bat Hp-betacoronavirus Zhejiang 2013.

In an embodiment, the immunogen used herein can be derived from a coronavirus spike RBD protein or a portion thereof from any viral species or strain in any one of the phylogenetically clustered clades of lineage B coronavirus (Sarbecovirus). For example, the coronavirus spike RBD protein or a portion thereof can be of any species or strain in clade 1, including but not limited to SARS-CoV, WIV1, LYRa11, Rs7327, Rs4231, Rs4084, and SHC014. The coronavirus spike RBD protein or a portion thereof can be of any species or strain in clade 2, including but not limited to, As6526, Yunnan 2011, Shaanxi 2011, 9-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, and 273-2005. The coronavirus spike RBD protein or a portion thereof can be of any species or strain in clade 1/2, including but not limited to SARS-CoV2. The coronavirus spike RBD protein or a portion thereof can be of any species or strain in clade 3, including but not limited to BM48-31.

In some embodiments, the immunogen used herein can be derived from a coronavirus spike RBD protein or a portion thereof can be from a coronavirus selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31.

In some embodiments, the immunogen used herein can, for example, can be derived from a coronavirus nucleocapsid protein (N protein) or a portion thereof. The N protein is a multifunctional RNA-binding protein required for viral RNA transcription, replication, and packaging. The N protein consists of three domains, an N-terminal RNA-binding domain, a central intrinsically disordered region, followed by a C-terminal dimerization domain. The RNA-binding domain contains multiple positively charged binding surfaces that form charged interactions with RNA promoting its helical arrangement. In some embodiments, the immunogen is derived from a coronavirus N protein or a portion thereof, comprising or consisting of an amino acid sequence having, having about, having at least, or having at least about, 80%, 85%, 90%, 95%, 98%, 99% or more, sequence identity to the amino acid sequence of any of the coronavirus N proteins from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31.

In some embodiments, the immunogen used herein can be derived from a coronavirus membrane protein (M protein) or a portion thereof. The M protein is the most abundant structural protein and defines the shape of the viral envelope. The M protein is regarded as the central organizer of the viral assembly, interacting with other major coronaviral structural proteins. In some embodiments, the immunogen is derived from a coronavirus M protein or a portion thereof comprising or consisting of an amino acid sequence having, having about, having at least, having at least about, 80%, 85%, 90%, 95%, 98%, 99% or more, sequence identity to the amino acid sequence of any of the coronavirus M proteins from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31.

In some embodiments, the immunogen used herein can be derived from a coronavirus envelope protein (E protein) or a portion thereof. The E protein is a small membrane protein and minor component of the virus particles. Without being bound to any theory, it is believed that the E protein plays roles in virion assembly and morphogenesis, alteration of the membrane of host cells, and virus-host cell interaction.

In some embodiments, the immunogen used herein can be derived from a coronavirus hemagglutinin-esterase protein (HE protein) or a portion thereof. The HE protein, which is an envelope protein, mediates reversible attachment to O-acetylated sialic acids by acting both as lectins and receptor-destroying enzymes. In some embodiments, the immunogen used herein can be derived from a coronavirus HE protein or a portion thereof comprising or consisting of an amino acid sequence having, having about, having at least, having at least about, 80%, 85%, 90%, 95%, 98%, 99% or more, sequence identity to the amino acid sequence of any of the coronavirus HE proteins from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31.

In some embodiments, the immunogen used herein can be derived from a coronavirus papain-like protease or a portion thereof. The coronavirus papain-like protease is one of several nonstructural proteins and is responsible for processing of viral proteins into functional, mature subunits during maturation. For example, the coronavirus papain-like protease can cleave a site at the amino-terminal end of the viral replicase region. In addition to its role in viral protein maturation, papain-like protease exhibits both a deubiquitinating and deISG15ylating activity. In vivo, this protease antagonizes innate immunity by acting on IFN beta and NF-kappa B signaling pathways. In some embodiments, the immunogen used herein can be derived from a coronavirus papain-like protease or a portion thereof comprising or consisting of an amino acid sequence having, having about, having at least, having at least about, 80%, 85%, 90%, 95%, 98%, 99% or more, sequence identity to the amino acid sequence of any of the coronavirus papain-like proteases from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31.

In some embodiments, the immunogen used herein can be derived from a coronavirus 3CL protease or a portion thereof. The 3CL protease is another main protease in addition to the papain-like protease and is required for processing of viral polypeptides into distinct, functional proteins.

In some embodiments, the 3CL protease is a SARS-CoV-2 3CL Protease, which is a C30-type cysteine protease located within the non-structural proteins 3 (NS3) region of the viral polypeptide. Analysis of the Coronavirus genome reveals at least 11 sites of cleavage for the 3CL protease, many containing the amino acid sequence LQ[S/A/G]. In some embodiments, the immunogen used herein can be derived from a coronavirus 3CL protease or a portion thereof comprising or consisting of an amino acid sequence having, having about, having at least, having at least about, 80%, 85%, 90%, 95%, 98%, 99% or more, sequence identity to the amino acid sequence of any of the coronavirus 3CL proteases from one or more coronaviruses selected from SARS-CoV, SARS-CoV2, WIV1, LYRa11, Rs7327, Rs4231, Rs4084, SHC014, As6526, Yunnan 2011, Shaanxi 2011, 279-2005, Rs4237, Rs4081, Rp3, Rs4247, HKU3-8, HKU3-13, GX2013, Longquan-140, YN2013, Rf4092, ZXC21, ZC45, JL2012, HuB2013, Rf1, HeB2013, 273-2005, and BM48-31.

In some embodiments, the plurality of coronavirus derived immunogens attached to a multivalent carrier can comprise coronavirus derived proteins of different protein types. For example, the plurality of coronavirus derived immunogens attached to a multivalent carrier can be derived from coronavirus S proteins or portions thereof as well as other coronavirus proteins such as a coronavirus N protein or a portion thereof, a coronavirus HE protein or a portion thereof, a coronavirus papain-like protease or a portion thereof, a coronavirus 3CL protease or a portion thereof, a coronavirus M protein or a portion thereof, or a combination thereof.

One or more of the plurality of coronavirus derived immunogens, or each of the plurality of coronavirus derived immunogens, can have a sequence identity of about, at least, or at least about 65%, 70%, 75%, 80%, 85%, 90%, 95%, 98%, or 99% with one another. In some embodiments, the plurality of coronavirus derived immunogens each comprise a coronavirus S protein RBD or a portion thereof, the coronavirus S protein RBDs or portions thereof having a sequence identity of about, at least, or at least about, 65%, 70%, 75%, 80%, 85%, 90%, 95%, 98%, or 99% with one another.

The embodiments disclosed herein focus on the coronavirus-derived immunogen portion presented by a nanoparticle carrier, but it is understood that these embodiments include at least one linear carbohydrate covalently attached to at least one immunogen in the plurality of immunogens presented by the nanoparticle carrier.

In another embodiment, the plurality of Borrelia derived immunogens covalently attached to a multivalent carrier can be of a same protein type or corresponding proteins. Borrelia derived immunogens of a same protein type may or may not have identical amino acid sequences, but generally share some sequence homology. For example, the Borrelia outer surface protein C (OspC) of different Borrelia strains are of a same protein type or corresponding proteins. As another example, the Borrelia outer surface protein A (OspA) proteins from different Borrelia strains are considered the same protein type or corresponding proteins. In some embodiments, proteins of different Borrelia taxonomic groups having the same function are considered the same protein type or corresponding proteins. In some embodiments, Borrelia immunogens of a same protein type have at least 50% sequence identity, for example at least 65%, 70%, 80%, 90%, 95%, 98%, 99%, or more sequence identity.

In one embodiment, the plurality of OspC types are associated with human Borrelia infection and are selected from the group comprising: T, U, B, E, K, H, N, C, and M; in further aspects, the plurality of OspC types are associated with human Borrelia infection and are selected from the group comprising: Pwa, Pli, PBes, Pki, PFim, Smar, HT22, A and K; and in yet further aspects, the plurality of OspC types are associated with canine Borrelia infection and are selected from the group comprising types I, H, N, C, M, D, and F.

In another embodiment, the plurality of Borrelia derived immunogens attached to a multivalent carrier can comprise Borrelia derived proteins of different protein types. For example, the plurality of Borrelia derived immunogens attached to a multivalent carrier can comprise OspC or portions thereof, as well as other Borrelia proteins such as OspA, OspB, OspE, or portions thereof. One or more of the plurality of Borrelia derived immunogens, or each of the plurality of Borrelia derived immunogens, can have a sequence identity of about, at least, or at least about 50%, 65%, 70%, 75%, 80%, 85%, 90%, 95%, 98%, or 99% with one another. In some embodiments, the plurality of Borrelia derived immunogens each comprise OspA or a portion thereof, OspC or a portion thereof, OspE or a portion thereof, and OspB or a portion thereof having a sequence identity of about, at least, or at least about, 50%, 65%, 70%, 75%, 80%, 85%, 90%, 95%, 98%, or 99% with one another.

One or more of the plurality of Babesia derived immunogens, or each of the plurality of Babesia derived immunogens, can have a sequence identity of about, at least, or at least about 50%, 65%, 70%, 75%, 80%, 85%, 90%, 95%, 98%, or 99% with one another. In some embodiments, the plurality of Babesia derived immunogens are derived from, but are not limited to, BMSA, BmSA1, BmSP44, BmPROF, BboPROF, BbigPROF, BmAMA-1, BmRON2, Bm2D41, BmSERA1, BmMCFPR1, BmPiβS1, BmBAHCS1, BboPROF, BdAMA1, BdPO, N-terminal and C-terminal fragments of BmRON2, Babesia microti methionine aminopeptidase protein 1, or a portion thereof having a sequence identity of about, at least, or at least about, 50%, 65%, 70%, 75%, 80%, 85%, 90%, 95%, 98%, or 99% with one another.

One or more of the plurality of Chlamydia derived immunogens, or each of the plurality of Chlamydia derived immunogens, can have a sequence identity of about, at least, or at least about 50%, 65%, 70%, 75%, 80%, 85%, 90%, 95%, 98%, or 99% with one another. In some embodiments, the plurality of Chlamydia derived immunogens are derived from, but are not limited to, OmpA, CPAF, PmpG, Chlamydia trachomatis OmpA serovars D, E, F, G, Chlamydia trachomatis CPAF, Chlamydia trachomatis PmpG, or a portion thereof having a sequence identity of about, at least, or at least about, 50%, 65%, 70%, 75%, 80%, 85%, 90%, 95%, 98%, or 99% with one another.

One or more of the plurality of Orthomyxoviridae derived immunogens, or each of the plurality of Picornaviridae derived immunogens, can have a sequence identity of about, at least, or at least about 50%, 65%, 70%, 75%, 80%, 85%, 90%, 95%, 98%, or 99% with one another. In some embodiments, the plurality of Orthomyxoviridae immunogens is derived from, but not limited to, VP1, VP2, VP3, and VP4, having a sequence identity of about, at least, or at least about, 50%, 65%, 70%, 75%, 80%, 85%, 90%, 95%, 98%, or 99% with one another.

One or more of the plurality of human immunodeficiency virus derived immunogens, or each of the plurality of human immunodeficiency virus derived immunogens, can have a sequence identity of about, at least, or at least about 50%, 65%, 70%, 75%, 80%, 85%, 90%, 95%, 98%, or 99% with one another. In some embodiments, the plurality of human immunodeficiency virus immunogens is derived from, but not limited to, ENV, GP160, Gag, Pol, Gag-Pol-Nef, having a sequence identity of about, at least, or at least about, 50%, 65%, 70%, 75%, 80%, 85%, 90%, 95%, 98%, or 99% with one another.

One or more of the plurality of Human orthopneumovirus derived immunogens, or each of the plurality of human immunodeficiency virus derived immunogens, can have a sequence identity of about, at least, or at least about 50%, 65%, 70%, 75%, 80%, 85%, 90%, 95%, 98%, or 99% with one another. In some embodiments, the plurality of Human orthopneumovirus immunogens is derived from, but not limited to RSVPreF3, having a sequence identity of about, at least, or at least about, 50%, 65%, 70%, 75%, 80%, 85%, 90%, 95%, 98%, or 99% with one another.

In order to facilitate the understanding of the present invention, the following definitions are provided:

A peptide, as defined by IUPAC, is amides derived from two or more amino carboxylic acid molecules (the same or different) by formation of a covalent bond from the carbonyl carbon of one to the nitrogen atom of another with formal loss of water. The term is usually applied to structures formed from α-amino acids, but it includes those derived from any amino carboxylic acid. One skilled in the art will recognize that said definition is independent of the stereochemistry within the polypeptide chain, and as such would be equally applicable to peptides and proteins derived from L-amino acids and D-amino acids. Polypeptides, as defined by IUPAC, are naturally occurring and synthetic peptides containing ten or more amino acid residues. Proteins, as defined by IUPAC, are naturally occurring and synthetic polypeptides having molecular weights greater than about 10,000 Da (the limit is not precise) (IUPAC, 1997).

As used herein, a serotype is defined as a variation within a microorganism species, distinguished by the humoral immune response. The serotype classification of bacteria or viruses is based on their surface antigens and was established before the availability of other techniques, such as genome sequencing or mass spectrometry. Antibodies generated to one serotype do not usually efficiently protect against another serotype. Serotypes have been described in many viral species and generally correspond to genotypes (Simon-Loriere et al, 2022).

As used herein, “sequence identity” or “identity” in the context of two nucleic acid or polypeptide sequences makes reference to the nucleotide bases or residues in the two sequences that are the same when aligned for maximum correspondence over a specified comparison window. Methods of alignment of sequences for comparison are well known in the art. Various programs and alignment algorithms are described in: Smith et al., 1981; Needleman et al., 1970; Pearson et al., 1988; Higgins et al., 1988; Higgins et al., 1989; Corpet, 1988; Huang et al., 1992; Pearson, 1994; and Altschul, 1990.

When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule. A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative, or neutral), and other properties identifiable to one of skill in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine. The term “substantially identical” as used herein in the context of two or more sequences refers to a specified percentage of amino acid residues or nucleotides that are identical or functionally equivalent, such as about, at least or at least about 65% identity, optionally, about, at least or at least about 70%, 75%, 80%, 85%, 90%, 95%, or 99% identity over a specified region or over the entire sequence.

As used herein, the term “variant” refers to a polynucleotide or polypeptide having a sequence substantially similar or identical to a reference (e.g., the parent) polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR), high throughput sequencing, Sanger sequencing, and hybridization techniques. Variant polynucleotides also include synthetically derived polynucleotides, such as those generated, for example, by using site-directed mutagenesis. Generally, a variant of a polynucleotide, including, but not limited to, a DNA, can have at least, or at least about, 50%, 55%, 60%, 65%, 70%, 75%, 80%, 85%, 90%, 91%, 92%, 93%, 94%, 95%, 96%, 97%, 98%, 99%, or more sequence identity to the reference polynucleotide as determined by sequence alignment programs known in the art. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot, Edman degradation, and mass spectroscopy. A variant of a polypeptide can have, for example, at least, or at least about, 60%, 65%, 70%, 75%, 80%, 85%, 90%, 91%, 92%, 93%, 94%, 95%, 96%, 97%, 98%, 99%, or more sequence identity to the reference polypeptide as determined by sequence alignment programs known in the art.

The multivalent or monovalent carriers herein described can be prepared using standard molecular biology procedures known to the person skilled in the art, as well as the protocols exemplified herein. In some embodiments, particle-forming subunits and/or the immunogens can be produced by liquid-phase or solid-phase chemical protein synthetic methods known to one of skill in the art. Production of the particle-forming subunits and/or the immunogens can use recombinant DNA technology well known in the art. For example, a tagged immunogen or immunogen functionalized with a protein tag can be synthesized using biosynthetic methods, such as cell-based or cell-free methods known to the person skilled in the art. A tagged immunogen can be produced using an expression vector comprising a nucleic acid molecule encoding the immunogen. The nucleic acid sequence encoding the particle-forming subunits and/or the immunogens can be operably linked to appropriate regulatory elements including, but not limited to, a promoter, enhancer, transcription initiation site, termination site, and translation initiation site. The vector can also comprise a nucleic acid molecule encoding one or more protein tags (e.g., a poly(His) tag, SpyTag). In some embodiments, the vector can additionally include a nucleic acid sequence encoding a trimerization motif (e.g., a foldon trimerization domain from T4 fibritin or viral capsid protein SHP). The vector can also comprise a nucleic acid sequence encoding a signal peptide that directs the protein into the proper cellular pathway, such as a signal peptide for secretion of the expressed protein into supernatant medium. The vector may comprise one or more selectable marker genes such as a gene providing ampicillin resistance or kanamycin resistance. Methods for the construction of nucleic acid constructs are well known. See, for example, Molecular Cloning: a Laboratory Manual, 3^{rd} edition and Current Protocols in Molecular Biology, 1994-1998. Protein biosynthesis of tagged immunogens can be performed by providing cell-based or cell-free protein translation systems with the expression vectors encoding the tagged immunogens. Similarly, a tagged particle-forming protein can be produced using an expression vector comprising a nucleic acid sequence encoding a particle-forming subunit and a nucleic acid sequence encoding a protein tag (e.g., SpyCatcher). In an exemplary embodiment, the multivalent carriers are produced following the protocols described in Cohen et al., 2021b.

In some embodiments, constructs expressing the nanoparticle carrier subunit and the immunogens can be introduced together into a host or transformation-competent cell. Multivalent carriers can be generated as a result of conjugation of the expressed immunogens to the self-assembled nanoparticles through a functional group pair or a reactive moiety pair described herein (e.g., SpyTag/SpyCatcher).

Nanoparticle Carriers (e.g., nanoparticles with SpyCatcher) and immunogens (e.g., SpyTagged protein immunogen) can, for example, be prepared separately and then incubated under a condition (e.g., in a TBS buffer at room temperature) for a certain time period (e.g., about, at least, or at least about 30 seconds, 1 minutes, 2 minutes, 3 minutes, 5 minutes, 10 minutes, 15 minutes, 30 minutes, 45 minutes, 1 hour, 2 hours, 5 hours, 10 hours, 12 hours, 15 hours, 24 hours, 48 hours, 72 hours, 96 hours) to allow for the conjugation of the nanoparticle carriers and the immunogen molecules.

In some embodiments, immunogen molecules are provided in an excess amount as compared to the particle-forming subunits of the nanoparticle carriers, such as 1-fold, 2-fold, 3-fold, 4-fold, 5-fold or greater than the particle-forming subunits.

A vaccine composition is a pharmaceutical composition that can elicit a prophylactic (e.g., to prevent or delay the onset of a disease, or to prevent the manifestation of clinical or subclinical symptoms thereof) or therapeutic (e.g., suppression or alleviation of symptoms) immune response in a subject. Provided herein is a vaccine composition comprising a monovalent or multivalent carrier as herein described. The vaccine composition can comprise one or more “pharmaceutically acceptable carriers.”

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent, or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) linear carbohydrates such as dextrans, chitin, partially deacylated chitin, chitosan, partially acylated chitosan, hyaluronic acid, keratin, keratin sulfate, chondroitin, chondroitin sulfate, dermatan, dermatan sulfate, and heparin, and derivatives thereof; and (22) other non-toxic compatible substances employed in vaccine formulations. In some embodiments, the pharmaceutically acceptable carrier is a carbohydrate (i.e., hyaluronic acid, partially deacylated chitin, chitosan, partially acylated chitosan), which may vary (i.e., molecular weight, functionalization) from the linear carbohydrate adjuvant. One of ordinary skill in the art would understand that one or more pharmaceutically acceptable carriers may be used in a vaccine composition.

In some embodiments, a pharmaceutically acceptable carrier comprises a pharmaceutical acceptable salt. As used herein, a “pharmaceutical acceptable salt” includes a salt of an acid from of one of the components of the compositions herein described. These include organic or inorganic acid salts of the amines. Preferred acid salts are hydrochlorides, acetates, salicylates, nitrates, and phosphates. Other suitable pharmaceutically acceptable salts are well known to those skilled in the art and include basic salts of a variety of inorganic and organic acids.

The vaccine composition can further comprise appropriate adjuvants. Adjuvant refers to any immunomodulating substance capable of being combined with the protein immunogens herein described to enhance, improve or otherwise modulate an immune response in a subject. Adjuvants include, but are not limited to, aluminum (AAHS, Alhydrogel®, aluminum hydroxide, aluminum phosphate, Alum), Quil-A®, Addavax™, CFA, AS03, AS04, MF59®, AS01_{B}, MPL®, CpG 1018, Poly I:C, Poly I:C12U, Poly I:CLC, Flagellin-based, Resiquimod-based (i.e., R848), GLA, Chitosan, LPS, Matrix-M™, Montanide ISA™51, and Montanide ISA™720. In another embodiment, a linear carbohydrate is an adjuvant, and also may be a pharmaceutically acceptable carrier, and/or a vehicle. That is, non-attached linear carbohydrate is part of the vaccine composition.

The vaccine composition can be formulated for a variety of modes of administration. Techniques for formulation and administration can be found, for example, in “Remington's Pharmaceutical Sciences.” In an embodiment, the vaccine compositions thereof can be administered to a subject systematically. The wording “systemic administration” as used herein indicates any route of administration by which a vaccine composition is brought in contact with the body of the individual, so that the resulting composition location in the body is systemic (i.e., not limited to a specific tissue, organ, or other body part where the vaccine is administered). Systemic administration can be continuous, chronic, short, or intermittent. In some embodiments, the vaccine compositions of the present disclosure may be specially formulated for administration in solid or liquid form, including those adapted for the following:
(1) Enteral administration, a route of administration wherein the vaccine composition is delivered via the digestive tract, and includes, but is not limited to, oral administration, administration by gastric feeding tube, administration by duodenal feeding tube, gastrostomy, enteral nutrition, and rectal administration. Enteral administration includes administration through the mouth, through a gastrostomy tube or jejunostomy tube if through a pre-programmed pump or through a syringe, or a metered dose inhaler. Enteral administration also includes, but is not limited to, drenches (aqueous or non-aqueous solutions or suspensions), tablets, boluses, powders, granules, pastes, pessaries, creams, foams, tablets, gels, or suppositories;
(2) Parenteral administration, a route of administration wherein the vaccine composition is delivered via injection or infusion, and includes, but is not limited to, subcutaneous administration, intravenous administration, intramuscular administration, intraarterial administration, intraperitoneal administration, or intracranial administration, i.e., intrathecal or intracerebroventricular administration. Parenteral administration includes, but not limited to, drenches (aqueous or non-aqueous solutions or suspensions), and boluses;
(3) Topical administration, a route of administration wherein the vaccine composition is delivered via, but not limited to, direct application to the external epidermis, dermis, mucous membrane, via buccal contact, via sublingual contact, or via ocular contact, but excludes intrapulmonary administration and intranasal administration. Topical administration includes, but is not limited to, application of creams, ointments, spray gels, lotions, via patches, via microneedles, or other formulations;
(4) Intravaginal administration, a route of administration wherein the vaccine composition is delivered inside the vagina. Intravaginal administration includes, but is not limited to, pessaries, creams, foams, tablets, gels, or suppositories; or,
(5) intranasal administration, a route of administration wherein the vaccine composition is delivered via intranasal and intrapulmonary administration. Intranasal administration to the body refers to the means of direct administration through the nostrils or the mouth resulting in contact of the vaccine composition with the nasal mucosa or other aspects of the nasal cavity. Intranasal administration includes, but is not limited to, an aqueous aerosol, liposomal preparation, inhalant, liquid drops, or other formulations that provide for contact of the vaccine composition with the mucosa.

Formulations useful in the methods of the present disclosure includes those suitable for enteral, parenteral, topical, intravaginal, intranasal, intrarectal, intraocular, and intravitreal, administration.

The formulations may conveniently be presented in unit dosage form and may be prepared by any methods well known in the art of pharmacy. The amount of active ingredient which can be combined with a pharmaceutically acceptable carrier to produce a single dosage form will vary depending upon the host being treated, and the particular mode of administration. The amount of active ingredient, which can be combined with a pharmaceutically acceptable carrier to produce a single dosage form will generally be that amount of the immunogen which produces a therapeutic effect or an immune response. Generally, out of one hundred percent, this amount will range from about 1% to about 99% of active ingredient, preferably from about 5% to about 70%, most preferably from about 5% to about 25%.

Formulations suitable for vaccine composition administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), and/or as mouth washes and the like.

In solid dosage forms for vaccine composition administration (capsules, tablets, pills, dragees, powders, granules and the like), the immunogen is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the vaccine compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for vaccine composition administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, and fatty acid esters of sorbitan, and mixtures thereof.

The vaccine composition can be presented in a unit dosage form, e.g., in ampoules or in multi-dose containers, with an optionally added preservative. The vaccine composition can further be formulated as suspensions, solutions, or emulsions in oily or aqueous vehicles, and may contain other agents including suspending, stabilizing and/or dispersing agents.

The vaccine composition can be formulated as an inhalant, liquid drops, aerosols, or other formulations. When administered as a liquid, compositions of the invention may be administered as an aqueous solution, e.g., a saline solution. The parameters of the formulation (e.g., pH, osmolarity, viscosity, etc.) may be adjusted as necessary to facilitate the delivery of the compositions of the invention.

The vaccine compositions disclosed herein can be employed in a variety of therapeutic or prophylactic applications to stimulate an immune response in a subject in need, to treat or prevent a pathogen-infection in a subject in need, and/or to treat or prevent a disease or disorder caused by a pathogen in a subject in need.

As used herein, the term “treatment” or “treat” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder, or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an occurrence or an event, including disease transmission and retransmission. Rather, it denotes that the likelihood of the occurrence of the event has been reduced in the presence of the compound or method.

The term “condition” as used herein indicates a physical status of the body of an individual (as a whole or as one or more of its parts), that does not conform to a standard physical status associated with a state of complete physical, mental, and social well-being for the individual. Conditions herein described include, but are not limited to, disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

The terms “subject”, “subject in need”, and “individual” as used herein refer to an animal. The term “animal” is intended to include mammals, birds, fish, amphibians, reptiles, and the like.

Animal or host includes humans and non-human mammals. Non-human mammals include, but are not limited to, Equidae (e.g., horse, zebra, asses), Canidae (e.g., dogs, wolves, foxes, coyotes, jackals), Felidae (e.g., domestic cats, wild cats including cheetahs, lions, tigers, leopard, and lynx), Bovidae (e.g., sheep, cattle, goats, buffalo, bison, wild oxen), Suidae (e.g., pig), Leporidae (e.g., rabbit), Primates (e.g., prosimian, tarsier, monkey, gibbon, ape, gorilla, chimpanzee, macaques, lemur), Rodentia (e.g. mouse, rat, hamster, marmot, squirrel, beavers, gerbils), Mustelae (e.g. weasel, ferret, mink), Cingulatae (e.g., armadillo), Chiropterae (e.g., bats), and Cervidae (e.g. deer, elk, moose). Other animals include, but are not limited to, Osteichthyes, Chondrichthyes, Aves (e.g., chicken, duck, goose, turkey, quail, pheasant, parrot, finches, hawk, crow, ostrich, emu, and cassowary), Amphibia (e.g., frogs, toads, newts, salamanders), and Reptilia (e.g., turtles, tuatara, lizards, snakes). The term “animal” also includes an individual animal in all stages of development, including embryonic and fetal stages.

In some embodiments, the subject or individual has been exposed to a pathogen. The term “exposed” indicates the subject has come in contact with a person or an animal that is infected with a pathogen. Exposed may also mean that the animal has come in contact with an environmental pathogen, or a pathogen borne by a host capable of infecting higher mammals, including humans. In some embodiments, a subject in need can be a healthy subject exposed to or at risk of being exposed to a pathogen. In some embodiments, subjects in need include those already suffering from the disease or disorder caused by a pathogen infection or those diagnosed with a pathogen infection.

Accordingly, the vaccine composition can be administered in advance of any symptom, for example, in advance of a pathogen infection. The vaccine composition can also be administered at or after the onset of a symptom of disease or infection, for example, after development of a symptom of infection or after diagnosis of the infection.

The phrase “therapeutically effective amount” as used herein means that amount of immunogen disclosed herein which is effective for producing some desired therapeutic effect and/or generating a desired response, such as reduction or elimination of a sign or symptom of a condition or disease, at a reasonable benefit/risk ratio. The therapeutically effective amount also varies depending on the structure and antigens of the immunogen, the route of administration utilized, and the specific diseases or disorders to be treated as will be understood to a person skilled in the art. For example, if a given clinical treatment is considered effective when there is at least a 20% reduction in a measurable parameter associated with a disease or disorder, a therapeutically effective amount of the immunogen—for the treatment of that disease or disorder is the amount necessary to achieve at least a 20% reduction in that measurable parameter.

In some embodiments, a therapeutically effective amount is necessary to inhibit pathogen replication or to measurably alleviate outward symptoms of the pathogen infection or inhibiting further development of the disease, condition, or disorder. In some embodiments, a therapeutically effective amount is an amount that prevents one or more signs or symptoms that can be caused by a pathogen-infection. In some embodiments, a therapeutically effective amount can be an amount that prevents one or more signs or symptoms of a particular disease or condition from developing, such as one or more signs or symptoms associated with pathogen infections.

A therapeutically effective amount of the vaccine composition herein described can be estimated from data obtained from cell culture assays and further determined from data obtained in animal studies, followed up by human clinical trials. For example, toxicity and therapeutic efficacy of the vaccine compositions described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred.

In some embodiments, the determination of a therapeutically effective amount of the vaccine composition can be measured by measuring the titer of antibodies produced against a pathogen.

Methods of determining antibody titers and methods of performing virus neutralization arrays are known to those skilled in the art.

In some embodiments, a method of stimulating an immune response in a subject in need is disclosed herein, the method comprising administering to the subject a pharmaceutically effective amount of the vaccine composition, thereby stimulating an immune response in the subject in need.

In some embodiments, administering the vaccine composition induces neutralizing responses against a pathogen.

In some embodiments, a method for treating or preventing a pathogen infection in a subject in need thereof is disclosed, the method comprising administering to the subject a pharmaceutically effective amount of the vaccine composition herein described, thereby treating or preventing the pathogen infection in the subject. In another embodiment, a method for treating an infection in a subject in need thereof is disclosed, the method comprising administering to the subject a pharmaceutically effective amount of the vaccine composition herein described, thereby improving the survival rate in the subject.

In some embodiments, a method for treating a pathogen infection in a subject in need thereof is disclosed, the method comprising administering to the subject a pharmaceutically effective amount of the vaccine composition herein described, thereby reducing the infectivity in the subject.

In each of the forgoing methods, the vaccine composition administration is independently and individually selected from the group comprising enteral, parenteral, topical, intravaginal, intranasal, intrarectal, intraocular, and intravitreal.

In some embodiments, the vaccine composition can be used for treating and preventing a broad spectrum of pathogen infections or a disease and disorder caused by such infections by inducing broadly protective anti-pathogen responses. For example, the vaccine composition herein described can elicit broadly neutralizing antibodies that neutralize one or more pathogens from a subfamily, genus, subgenus, species, and/or strain that differ from the subfamily, genus, subgenus, species, and/or strain of the pathogens from which the pathogen antigens are derived to produce the vaccine composition.

The vaccine composition herein disclosed can be administered to a subject using a single dose or a prime/boost protocol. In an embodiment, the methods herein described can comprise administrating to a subject a vaccine composition once. In another embodiment, the vaccine composition described herein can be administered to the subject in need two or more times. For example, the methods herein described can comprise administering to the subject a first vaccine composition, and after a period of time, administering to the subject a second vaccine composition. In a prime/boost protocol, a first vaccine composition is administered to the subject (prime) and then after a period of time, a second vaccine composition can be administered to the subject (boost). Administration of the second composition (boost composition) can occur days, weeks, months, or years after administration of the first composition (prime composition). For example, the boost composition can be administered about three days, 1 week, 2 weeks, 3 weeks, 4 weeks, 5 weeks, 6 weeks, 7 weeks, 8 weeks, 10 weeks, 12 weeks, 16 weeks, 20 weeks, 24 weeks, 28 weeks, 6 months, 1 year, 5 years, 10 years, or a number or a range between any two of these values, after the prime composition is administered.

The prime vaccine composition and the boost vaccine composition can be, but need not be, the same composition. In some embodiments, the prime vaccine composition and the boost vaccine composition can contain the same or different immunogens. In some embodiments, the prime vaccine composition and the boost vaccine composition can contain the same immunogens, but in different pharmaceutically effective amounts. In some embodiments, the prime vaccine composition and the boost vaccine composition can contain different adjuvants (i.e., different sized linear carbohydrates). In some embodiments the prime vaccine composition and the boost vaccine composition, can be administered by the same method (i.e., both intranasal) or administrated by different methods (i.e., parenteral and intranasal, or enteral and intranasal). In some embodiments, the boost vaccine composition can be administered more than once.

In some embodiments, the boost composition may be comprised of linear carbohydrate with optional vehicles for formulation purposes. That is, the boost composition is free of immunogen and monovalent or multivalent carriers.

In some embodiments, the vaccine compositions may also be used in order to prepare antibodies, both polyclonal and monoclonal, for, e.g., diagnostic purposes, as well as for immunopurification of the antigen of interest. If polyclonal antibodies are desired, a selected mammal, (e.g., mouse, rabbit, goat, horse, etc.) is immunized with the vaccine compositions of the present invention (prime) as described above. The animal optionally receives a boost 2-6 weeks later with one or more administrations of the vaccine compositions of the present invention as described above.

Polyclonal antisera is then obtained from the immunized animal and treated according to known procedures (see, e.g., Jürgens et al., 1985).

The nanoparticle carrier, glycosylated immunogens, and the vaccine composition containing the nanoparticle carrier as described herein can be provided as components of a kit.

Kits can include monovalent or multivalent carriers or vaccines of the present disclosure as well components for making such monovalent or multivalent carriers and vaccines. As such, kits can include, for example, primers, nucleic acid molecules, expression vectors, nucleic acid constructs encoding protein immunogens and/or particle-forming subunits described herein, cells, buffers, substrates, reagents, administration means (e.g., syringes), and instructions for using any of said components. Kits can also include pre-formed nanoparticle carriers, immunogens, glycosylated immunogens, and linear carbohydrates functionalized with an oxazoline herein described. It should be appreciated that a kit may comprise more than one container comprising any of the aforementioned, or related, components. For example, certain parts of the kit may require refrigeration, whereas other parts can be stored at room temperature. Thus, as used herein, a kit can comprise components sold in separate containers by one or more entity, with the intention that the components contained therein be used together.

Structural, chemical and stereochemical definitions are broadly taken from IUPAC recommendations, and more specifically from Glossary of Terms used in Physical Organic Chemistry (IUPAC Recommendations 1994) as summarized by Müller, P. Pure Appl. Chem. 1994, 66, pp. 1077-1184 and Basic Terminology of Stereochemistry (IUPAC Recommendations 1996) as summarized by Moss, G. P. Pure Appl. Chem. 1996, 68, pp. 2193-2222.

For convenience, certain terms employed in the specification, examples and claims are collected here. Unless defined otherwise, all technical and Scientific terms used in this disclosure have the same meanings as commonly understood by one of ordinary skill in the art to which this disclosure belongs. The initial definition provided for a group or term provided in this disclosure applies to that group or term throughout the present disclosure.

The term “tautomer” as used herein refers to compounds produced by the phenomenon where in a proton of one atom of a molecule shifts to another atom. See March, Advanced Organic Chemistry: Reactions, Mechanisms and Structures, 4th Ed., John Wiley & Sons, pp. 69-74 (1992). Tautomerism is defined as isomerism of the general form:

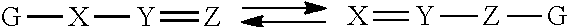

where the isomers (called tautomers) are readily interconvertible; the atoms connecting the groups X, Y and Z are typically any of C, H, O, or S, and G is a group which becomes an electrofuge or nucleofuge during isomerization. The most common case, when the electrofuge is H⁺, is also known as “prototropy”. Tautomers are defined as isomers that arise from tautomerism, independent of whether the isomers are isolable.

Oligosaccharides are available in a range of molecular weights. Linear oligosaccharides based on repeating monosaccharide or disaccharide subunits, such as hyaluronic acid and the other glycosaminoglycans (Yeung et al., 2002, Roth et al., 2008, Yamada et al., 2011), may be obtained as discreet sized species or a distribution of sizes about an average molecular weight. Other linear oligosaccharides include chitin, and partially deacetylated chitin (Cho et al., 2000, Cheung et al., 2015). The latter refers to chitin that has been processed to remove some or most of the N-acetyl moieties that modify the glucosamine core. The degree of deacetylation refers to the percentage of N-acetyl moieties removed, with up to 95% removal possible to retain the parent chitin identity. Removal of at least 49% of the acetyl groups within the polymer molecule is required for solubility in water. A range of molecular weights of the parent chitin and the partially deacetylated chitins are possible.

The molecular weight of a linear carbohydrate may be obtained from a variety of direct and indirect physical characterization methods. These include, but are not limited to, high performance liquid chromatography (HPLC), gel permeation chromatography (GPC), size-exclusion chromatography (SEC), mass spectroscopy (MS) including matrix assisted laser desorption ionization mass spectroscopy (MALDI-MS), ultracentrifuge sedimentation, viscosity, osmotic pressure, and dynamic light scattering (DLS), all of which may be affected by the number, size, or shape of molecules in a matrix, a suspension, or in a solution (Orviský et al., 1991, Motohashi et al., 1984, Yeung et al., 1999, Harding et al., 1991).

For distributions of different sized linear carbohydrates, weight-average molecular weight (Mw) and molecular weight distributions may be determined from ultracentrifuge sedimentation, diffusion, and light scattering. Number-average molecular weight (Mn) and molecular weight distributions may be determined from osmotic pressure and intrinsic viscosity determinations. Optical properties are best reflected in the weight-average molecular weight, while strength properties are best reflected in number-average molecular weight. Mn is the number of monomer molecules divided by the total number of molecules times the monomer mole weight. Mw is the area under the weight distribution curve that is divided into two equal parts. Mn<=Mw.

Weight-average molecular weight, is calculated by the equation

$${M}_{w} = \{ Sum [ ( {W}_{i} ) {( MW )}_{i} ] \} / \{ Sum {W}_{i} \} ,$$

where Wᵢ is the weight fraction of each size fraction and (MW)ᵢ is the mean molecular weight of the size fraction. The “weight-average” molecular weight is particularly significant in the analysis of properties such as viscosity, where the weight of the molecules is important. Number-average molecular weight is calculated by the equation

$${M}_{n} = \{ Sum [ ( {W}_{i} ) {( MW )}_{i} ] \} / \{ Sum {X}_{i} \} ,$$

where the value Xᵢ is the number of molecules in each size fraction.

The following examples set forth preferred methods in accordance with the invention. It is understood, however, that these examples are provided by way of illustration, and nothing therein should be taken as a limitation upon the overall scope of the invention.

Protein expression and purification: Proteins were either purchased from commercial vendors or prepared by standard recombinant over-expression techniques using published methods (Eschenfeldt et al., 2009) in E. Coli. (Ausubel, 2003), or Pichia Pastoris (Zonneveld et al., 1995, Ahmad et al., 2014), and purified by standard methods (Qiagen, 2003).

Carbohydrates: N-acetylglucosamine (GlcNAc), N-acetylmannosamine (ManNAc), N-acetylgalactosamine (GalNAc), diacetylchitobiose (DACB), triacetylchitotriose (TACT), and hyaluronic acid polymers of various lengths including those of less than 6K Mw (HyA6), those of less than 25K Mw (HyA25), those of less than 50K Mw (HyA50), and those of less than 110K Mw (HyA110) were obtained from commercial sources. Other linear carbohydrates are available either from commercial sources or by isolation and purification form natural sources. All samples were screened for the absence of LPS.

Example 1—General oxazoline formation protocol: A carbohydrate with an N-acyl-2-amino moiety at the reducing end was dissolved in deionized H₂O. At least 7.5 molar equivalents of triethylamine or Na₃PO₄ were added until the pH was 11. Three molar equivalents of either DMC or CDMBI were added and the reaction incubated at 4° C. from 2 to 24 hours. The pH of the solution was then adjusted to pH 8-8.5 with 1 M bicine or to pH 6, 7, or 8 with 1M MES. After adjustment of pH, some oxazolines were flash frozen in liquid nitrogen and stored at −80° C. until use. CDMBI (2-chloro-1,3-dimethyl-1H-benzimidazol-3-ium chloride) was prepared according to the method of Noguchi et al., 2012. DMC (chloro-1,3-dimethylimidazolinium chloride) was purchased from commercial sources.

Example 2—General preparation of protein: Protein was prepared for conjugation by either dissolving lyophilized protein in PBS, pH 8.0 followed by desalting via a G-50 spin column equilibrated with PBS pH 8.0, or dissolved in PBS, pH 8.0, and used directly. The protein concentration was determined by triplicate Bradford assay (Bradford, 1976). The volume of solution containing from about 5 μg to 50 mg was aliquoted into a microfuge tube for use in the conjugation reaction.

Example 3—General conjugation of neutralized oxazoline carbohydrate with a protein: The neutralized oxazolines and target proteins were prepared as above. The protein solution was dispensed into a microfuge tube followed by addition of about a 100 to 1000-fold molar excess of the carbohydrate oxazoline and allowed to react from 2 minutes to 72 hours at 4° C. or room temperature. Up to 5 ug samples were withdrawn for SDS-PAGE gel mobility shift assay to determine reaction completeness. Novex 8% and/or 12% bis-tris SDS-PAGE gels were used with a MES-SDS running buffer. The remainder of the samples were then cryopreserved by addition of glycerol to 20% v/v, flash frozen in liquid nitrogen, and stored at −80° C. SDS-PAGE gels were stained with PageBlue (Thermo Scientific) and destained in deionized water optionally supplemented with 1% acetic acid (Ausubel, 2003). Destained gels were visualized on an optical trans-illuminator, and the relative gel mobility evaluated based on the lagging edge of each band.

Conjugated constructs derived from highly charged carbohydrate oxazolines (i.e., hyaluronic acid, the glycosaminoglycans, partially deacetylated chitin) (Yeung et al., 2002, Roth et al., 2008, Yamada et al., 2011) may produce distortion in the band morphology, wherein the band morphology being manifest as distortion, streaking, smearing, lengthening, lightening of the stained band, formation of a chevron-shaped and/or U-shaped band, and/or extension of the band wherein the distal portions of the band display retarded mobility relative to the center of the band in the lane, and wherein the change in morphology is relative to the unconjugated protein sample band (Asubel, 2003, Osset et al., 1989, Møller et al., 2009, See et al., 1985).

Example 4-5 mg of GlcNAc was initially resuspended in 500 ul of deionized water. After GlcNAc was dissolved, 23.6 ul of triethylamine and 14.65 mg of CDMBI were added and mixed by inversion and held at 4° C. overnight. The next day, 100 ul of 1 M bicine, pH 8 was added to bring the pH to 8-8.5. The total volume was 1.105 ml and the GlcNAc concentration was 22.62 mg/ml.

Example 5-1 mg of TACT (N,N′,N″-Triacetylchitotriose) was initially resuspended in 25 ul of deionized water. After TACT was completely dissolved, 1.66 ul of triethylamine and 1.03 mg of CDMBI were added and mixed by inversion and held at 4° C. overnight. The next day, 25 ul of 1 M bicine, pH 8 was added to bring the pH to 8-8.5. The total volume was 52 ul and the TACT concentration was 19.36 mg/ml.

Example 6-5 mg of HyA6 (hyaluronic acid less than 6 kDa) was initially resuspended in 100 ul of deionized water. After HyA6 was completely dissolved, 1.74 ul of triethylamine and 1.08 mg of CDMBI were added and mixed by inversion and held at 4° C. overnight. The next day, 50 ul of 1 M bicine, pH 8 was added to bring the pH to 8-8.5. The total volume was 152 ul and the HyA6 concentration was 32.95 mg/ml.

Example 7-50 mg of HyA25 (hyaluronic acid less than 30 kDa) was initially resuspended in 1 ml of deionized water. After HyA25 was completely dissolved, 10 ul of triethylamine and 1.3 mg of CDMBI were added and mixed by inversion and held at 4° C. overnight. The next day, 150 ul of 1 M bicine, pH 8 was added to bring the pH to 8-8.5. The total volume was 1.16 ml and the HyA25 concentration was 43.10 mg/ml.

Example 8-50 mg of HyA50 (hyaluronic acid less than 50 kDa) was initially resuspended in 1 ml of deionized water. After HyA50 was completely dissolved, 10 ul of triethylamine and 1.3 mg of CDMBI were added and mixed by inversion and held at 4° C. overnight. The next day, 150 ul of 1 M bicine, pH 8 was added to bring the pH to 8-8.5. The total volume was 1.16 ml and the HyA50 concentration was 43.10 mg/ml.

Example 9-25 mg of HyA110 (hyaluronic acid less than 110 kDa) is initially resuspended in 1 ml of deionized water. After HyA110 is completely dissolved, 5 ul of triethylamine and 3 mg of CDMBI is added and mixed by inversion and held at 4° C. overnight. The next day, 1 M bicine (pH 8) is added to bring the pH to 8-8.5. The HyA110 concentration is determined.

Example 10—OspA-GST protein from NiCl₂ purification was desalted into PBS using G-50 Sephadex spin column. The protein was at a concentration of 1.4 mg/ml. The OspA-GST carbohydrate conjugations were combined with an approximate 1:100 (protein to carbohydrate molar ratio).

Example 11-270 ul (100 ug) OspA-GST was combined with 4 ul (31 ug) GlcNAc oxazoline. This was then incubated overnight at room temperature.

Example 12-1 ul (10 ug) OspA-GST was combined with 4 ul (31 ug) TACT oxazoline. This was then incubated overnight at room temperature.

Example 13-50 ul (50 ug) OspA-GST was combined with 11.9 ul (214 ug) HyA6 oxazoline. This was then incubated overnight at room temperature.

Example 14-20 ul (200 ug) OspA-GST was combined with 580 ul (25 ug) HyA25 oxazoline. This was then incubated overnight at room temperature.

Example 15-10 ul (100 ug) OspA-GST was combined with 290 ul (12.5 ug) HyA50 oxazoline. This was then incubated overnight at room temperature.

Example 16-10 ul (100 ug) OspA-GST is combined with 290 ul (12.5 ug) HyA110 oxazoline. This is then incubated overnight at room temperature.

Example 17—SHC104-RBD was produced according to the procedures of Cohen et al., 2021a and Keeble et al., 2019. SHC104-RBD was diluted to 10 mg/ml. The SHC104-RBD: carbohydrate conjugations were combined with an approximate 1:100 (protein to carbohydrate molar ratio).

Example 18-5 ul (50 ug) SHC104-RBD was combined with 7 ul (54 ug) GlcNAc oxazoline. This was then incubated overnight at room temperature.

Example 19-1 ul (10 ug) SHC104-RBD was combined with 4 ul (33 ug) TACT oxazoline. This was then incubated overnight at room temperature.

Example 20-50 ul (50 ug) SHC104-RBD was combined with 11.9 ul (214 ug) HyA6 oxazoline. This was then incubated overnight at room temperature.

Example 21-20 ul (200 ug) SHC104-RBD was combined with 580 ul (25 ug) HyA25 oxazoline. This was then incubated overnight at room temperature.

Example 22-10 ul (100 ug) SHC104-RBD was combined with 290 ul (12.5 ug) HyA50 oxazoline. This was then incubated overnight at room temperature.

Example 23-10 ul (100 ug) SHC104-RBD is combined with 290 ul (12.5 ug) HyA110 oxazoline. This is then incubated overnight at room temperature.

Example 24—Mosaic-8 RBD mi3 nanoparticle was produced according to the procedures of Cohen et al., 2021a. Dry mosaic-8 RBD mi3 nanoparticle, previously lyophilized in PBS, was resuspended in deionized water with 0.75% CHAPS to a concentration of 1 mg/ml. The mosaic-8 RBD mi3 nanoparticle: carbohydrate conjugations were combined with an approximate 1:50 (protein to carbohydrate molar ratio).

Example 25-270 ul (100 ug) mosaic-8 RBD mi3 nanoparticle was combined with 2 ul (15 ug) GlcNAc oxazoline. This was then incubated overnight at room temperature.

Example 26-270 ul (100 ug) mosaic-8 RBD mi3 nanoparticle was combined with 6 ul (46.5 ug) TACT oxazoline. This was then incubated overnight at room temperature.

Example 27-50 ul (50 ug) mosaic-8 RBD mi3 nanoparticle was combined with 11.9 ul (214 ug) HyA6 oxazoline, 5 ul 7.5% CHAPS, and 33.1 ul PBS. This was then incubated overnight at room temperature.

Example 28-200 ul (200 ug) mosaic-8 RBD mi3 nanoparticle was combined with 150.71 ul (3571 ug) HyA25 oxazoline, 20 ul 7.5% CHAPS, and 29.3 ul PBS. This was then incubated overnight at room temperature.

Example 29-100 ul (100 ug) mosaic-8 RBD mi3 nanoparticle was combined with 75.36 ul (1786 ug) HyA50 oxazoline, 10 ul 7.5% CHAPS, and 14.64 ul PBS. This was then incubated overnight at room temperature.

Example 30-100 ul (100 ug) mosaic-8 RBD mi3 nanoparticle is combined with 75.36 ul (1786 ug) HyA110 oxazoline, 10 ul 7.5% CHAPS, and 14.64 ul PBS. This is then incubated overnight at room temperature.

Example 31—For each of eight OspC variants included in the mosaic-8-mi3 nanoparticle, a synthetic double stranded DNA fragment encoding the OspC variant gene+a sequence encoding a 6×his tag+a sequence encoding a SpyTag or SpyTag derivative sequence at either the N- or C-terminal region of the OspC coding region with vector targeting sequences at the 5′ and 3′ ends, was purchased from a commercial supplier. The DNA fragment was then cloned by either Gibson assembly or megaprimer PCR mutagenesis into a T7 promotor driven bacterial expression vector. OspC variant expression vectors were then independently transformed into BL21(DE3) E. coli and each protein expressed in shake flask cultures. OspC SpyTag variants were purified from cellular lysates by immobilized metal ion affinity chromatography and the OspC SpyTag variant eluted with 300 mM imidazole containing column buffer. Each OspC Spytag variant was subsequently purified to homogeneity by size exclusion chromatography.

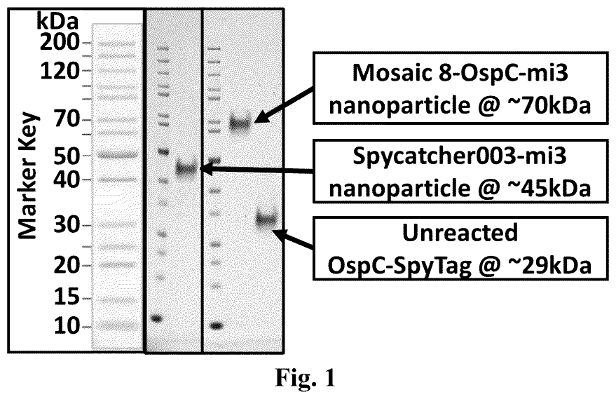

Example 32—Mosaic-8-OspC-mi3 nanoparticle was prepared by a method derivative of that employed by Tan et al., 2021. Final assembly of the mosaic-8-OspC mi3 nanoparticle was accomplished by incubating all 8 OspC SpyTag variants together with a sub stochiometric amount of the SpyCatcher003-mi3 nanoparticle precursor protein based on the total concentration of SpyTag subunits. Assembled mosaic-8-OspC mi3 nanoparticle was separated from free OspC-SpyTag variant proteins by high resolution size exclusion chromatography and ion exchange chromatography. See FIG. 1.

Example 33—Homotypic OspC mi3 nanoparticle was prepared by a method derivative of that employed by Tan et al., 2021. Final assembly of the homotypic-OspC mi3 nanoparticle was accomplished by incubating a single variant of OspC with the SpyTag from Example 31 together with a sub stochiometric amount of the SpyCatcher003-mi3 nanoparticle precursor protein based on the total concentration of SpyCatcher subunit. Assembled homotypic OspC mi3 was separated from the free OspC-SpyTag protein by high resolution size exclusion chromatography and ion exchange chromatography.

Example 34—For each of eight OspC variants included in the mosaic-8-Osp mi3 nanoparticle, a synthetic double stranded DNA fragment encoding the OspC variant gene+a sequence encoding a 6×his tag targeting sequences at the 5′ and 3′ ends, was purchased from a commercial supplier.

The DNA fragment was then cloned by either Gibson assembly or megaprimer PCR mutagenesis into a T7 promotor driven bacterial expression vector. OspC variant expression vectors were then independently transformed into BL21(DE3) E. coli and each protein expressed in shake flask cultures. OspC variants were purified from cellular lysates by immobilized metal ion affinity chromatography and the OspC variant eluted with 300 mM imidazole containing column buffer. Each OspC variant was subsequently purified to homogeneity by size exclusion chromatography.

Example 35—HyA50 kDa was dissolved to 3-5% w/v in ddi H₂O and 9 molar equivalents triethylamine (TEA) and 3 molar equivalents of 2-Chloro-1,3-dimethyl-1H-benzimidazol-3-ium chloride (CDMBI) or 2-Chloro-1,3-dimethylimidazolinium Chloride (DMC) were added sequentially and oxazoline formation was allowed to proceed at 4° C. for 30 minutes to 16 hours. 1 M bicine (pH 8.0) was then titrated until the pH of the reaction was between 8.0 and 8.5. A solution of mosaic-8-OspC mi3 nanoparticle was then prepared for reaction with the HyA50 oxazoline by application to a gel filtration column equilibrated with a buffer comprised of 10 mM Bicine buffer pH 8.15+140 mM NaCl. After the protein concentration was determined by Bradford assay or other protein concentration assay, the protein was combined with the HyA50 oxazoline derivative at a molar ratio between 1:1 to 1:10000 (protein: HyA50 oxazoline) and allowed to react in the dark at room at room temperature for 24 hr. Separation of HyA50 functionalized mosaic-8-OspC mi3 nanoparticle and the unreacted HyA50 oxazoline derivative was performed, if necessary, on a high-resolution gel filtration column.

Example 36—HyA110 is attached to the mosaic-8-OspC mi3 nanoparticle using the method described in Example 35.

Example 37—HyA50 was attached to the homotypic OspC mi3 nanoparticle using the method described in Example 35.

Example 38—HyA50 is attached to each individual OspC variant protein using the method described in Example 35.

Example 39—Genes for the production of soluble recombinant trimeric hemagglutinin (HGA) and soluble recombinant tetrameric neuraminidase (NA) proteins lacking transmembrane domains and the production of soluble recombinant trimeric hemagglutinin (HGA) and soluble recombinant tetrameric neuraminidase (NA) proteins lacking transmembrane domains with the SpyTag domain are created as described in Example 31. Production of soluble influenza vaccine immunogens derived from HGA and NA, are manufactured by recombinant overexpression in either stably or transiently transfected mammalian cell lines according to the methods described by Ecker et al., 2020. Open reading frames of proteins are purified from the resulting cultures by contemporary protein chromatography methods such as immobilized metal ion affinity chromatography, immunoaffinity chromatography, size exclusion chromatography, and ion exchange chromatography. Non-SpyTag HyA and HGA immunogen variants are also produced using similar methods.

Example 40—Mosaic-8-NA-HGA-mi3 nanoparticle is prepared as described in Example 32.

Example 41—Homotypic NA and HGA-mi3 nanoparticles are prepared as described in Example 33.

Example 42—HyA50 is attached to the mosaic-8-NA-HGA mi3 nanoparticle using the method described in Example 35.

Example 43—HyA50 is attached to the homotypic NA and HGA mi3 nanoparticles using the method described in Example 35.

Example 44—HyA50 is attached to individual NA and HGA proteins using the method described in Example 35.

Example 45—Genes for the production of soluble Babesia immunogens are taken from the list including, but not limited to, BMSA, BmSA1, BmSP44, BmPROF, BboPROF, BbigPROF, BmAMA-1, BmRON2, Bm2D41, BmSERA1, BmMCFPR1, BmPiβS1, BmBAHCS1, BboPROF, BdAMA1, BdPO, N-terminal and C-terminal fragments of BmRON2, B. microti heat-shock protein-70, Babesia microti methionine aminopeptidase protein 1 with the SpyTag domains, are created as described in Example 31. Production of SpyTag Babesia immunogens is accomplished using standard methods. Open reading frames of proteins are purified from the resulting cultures by contemporary protein chromatography methods such as immobilized metal ion affinity chromatography, immunoaffinity chromatography, size exclusion chromatography, and ion exchange chromatography. Non-SpyTag Babesia immunogen variants are also produced using similar methods.

Example 46—Mosaic-8-Babe-mi3 nanoparticle is prepared as described in Example 32.

Example 47—Homotypic Babe-mi3 nanoparticles are prepared as described in Example 33.

Example 48—HyA50 is attached to the mosaic-8-Babe-mi3 nanoparticle using the method described in Example 35.

Example 49—HyA50 is attached to the homotypic Babe-mi3 nanoparticles using the method described in Example 35.

Example 50—HyA50 is attached to the individual Babesia immunogenic proteins using the method described in Example 35.

Example 51—Genes for the production of Chlamydia immunogens are taken from the list including, but not limited to, MOMP serovars D, E, F, G, CPAF, PMPG with the SpyTag domains, are created as described in Example 31. Production of SpyTag Chlamydia immunogens is accomplished using standard methods. Open reading frames of proteins are purified from the resulting cultures by contemporary protein chromatography methods such as immobilized metal ion affinity chromatography, immunoaffinity chromatography, size exclusion chromatography, and ion exchange chromatography. Non-SpyTag Chlamydia immunogen variants are also produced using similar methods.

Example 52—Mosaic-6-Chly-mi3 nanoparticle is prepared as described in Example 32.

Example 53—Homotypic Chly-mi3 nanoparticles are prepared as described in Example 33.

Example 54—HyA50 is attached to the mosaic-6-Chly-mi3 nanoparticle using the method described in Example 35.

Example 55—HyA50 is attached to the homotypic Chly-mi3 nanoparticles using the method described in Example 35.

Example 56—HyA50 is attached to the individual Chlamydia immunogenic proteins using the method described in Example 35.

Example 57—Genes for the production of HIV immunogens are taken from the list including, but not limited to, ENV, GP160, Gag, -Pol, -Nef with the SpyTag domains, are created as described in Example 31. Production of SpyTag HIV immunogens is accomplished using standard methods.

Open reading frames of proteins are purified from the resulting cultures by contemporary protein chromatography methods such as immobilized metal ion affinity chromatography, immunoaffinity chromatography, size exclusion chromatography, and ion exchange chromatography. Non-SpyTag HIV immunogen variants are also produced using similar methods.

Example 58—Mosaic-HIV-mi3 nanoparticle is prepared as described in Example 32.

Example 59—Homotypic HIV-mi3 nanoparticles are prepared as described in Example 33.

Example 60—HyA50 is attached to the mosaic-HIV-mi3 nanoparticle using the method described in Example 35.

Example 61—HyA50 is attached to the homotypic HIV-mi3 nanoparticles using the method described in Example 35.

Example 62—HyA50 is attached to the individual HIV immunogenic proteins using the method described in Example 35.

Example 63—Genes for the production of the RSV immunogens are taken from the list including, but not limited to, RSVPreF3 with the Spytag domains, are created as described in Example 31.

Production of SpyTag RSV immunogens is accomplished using standard methods. Open reading frames of proteins are purified from the resulting cultures by contemporary protein chromatography methods such as immobilized metal ion affinity chromatography, immunoaffinity chromatography, size exclusion chromatography, and ion exchange chromatography. Non-SpyTag RSV immunogen variants are also produced using similar methods.

Example 64—Mosaic-RSV-mi3 nanoparticle is prepared as described in Example 32.

Example 65—Homotypic RSV-mi3 nanoparticles are prepared as described in Example 33.

Example 66—HyA50 is attached to the mosaic-RSV-mi3 nanoparticle using the method described in Example 35.

Example 67—HyA50 is attached to the homotypic RSV-mi3 nanoparticles using the method described in Example 35.

Example 68—HyA50 is attached to the individual RSV immunogenic proteins using the method described in Example 35.

Example 69—Genes for the production of the rhinovirus immunogens are taken from the list including, but not limited to, VP1, VP2, VP3, VP4 with the SpyTag domains, are created as described in Example 31. Production of SpyTag rhinovirus immunogens is accomplished using standard methods. Open reading frames of proteins are purified from the resulting cultures by contemporary protein chromatography methods such as immobilized metal ion affinity chromatography, immunoaffinity chromatography, size exclusion chromatography, and ion exchange chromatography. Non-SpyTag rhinovirus variants are also produced using similar methods.

Example 70—Mosaic-Rhino-mi3 nanoparticle is prepared as described in Example 32.

Example 71—Homotypic Rhino-mi3 nanoparticles are prepared as described in Example 33.

Example 72—HyA50 is attached to the mosaic-Rhino-mi3 nanoparticle using the method described in Example 35.

Example 73—HyA50 is attached to the homotypic Rhino-mi3 nanoparticles using the method described in Example 35.

Example 74—HyA50 is attached to the individual Rhino immunogenic proteins using the method described in Example 35.

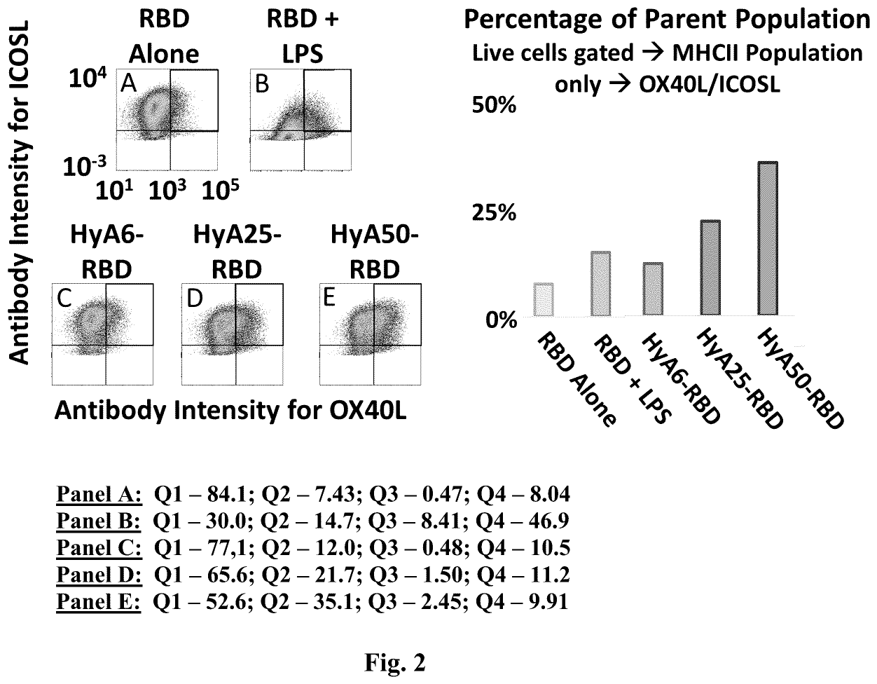

Example 75—Roughly 106 BMDCs were used for each experiment. Femurs and tibia bones from 8-12-week-old BALB/c mice were removed, and bone marrow collected into a sterile petri dish containing RPMI media+FBS (10%)+Pen/Strep (complete RPMI). Cells were monodispersed using a pipette, counted, and centrifuged at 300×G for 10 min. Cells were resuspended in complete RPMI with 20 ng/mL of GM-CSF. 2.5×10⁶ cells and added to each well of a 12-well plate and maintained at 37° C. with 5% CO₂. On Day 2, 70% of media was removed and replaced. On Day 3, 2 μg of the HyA-immunogen complex was then added to each set of cells. On Day 5, the cells were stained for appropriate cell markers and fixed in 2% paraformaldehyde in PBS. Following 48 hours of exposure to the HyA-immunogen, cells were analyzed using flow cytometry to determine the amount of surface expression of maturation markers known to correlate to strong T_{FH} responses including MHC-II, CD80, CD86, OX40L, and ICOSL. Flow cytometry was accomplished using a BD FACSAria IIIu and FlowJo software for analysis. See FIG. 2.

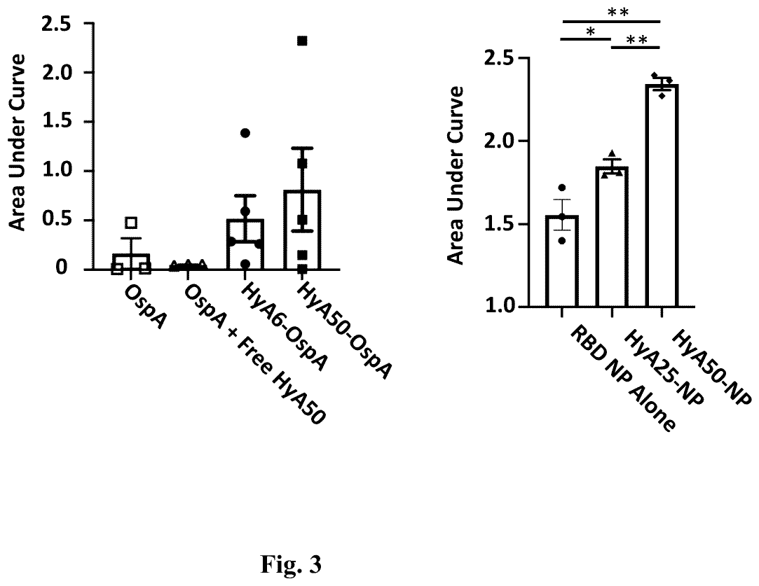

Example 76—Female BALB/c mice (n=5 to 10/cohort) were administered a 5 ug protein equivalent injected IM as a bolus, followed by a 5 ug IM booster injection at 4 weeks post-prime. Blood was collected via submandibular bleed at 4 and 8 weeks post-prime injection and the serum was analyzed for IgG against the OspA protein using standard ELISA techniques. Cohorts were immunized with the Osp-A protein covalently to HyA (HyA 6 or HyA50), the OspA protein alone, and OspA combined with, but not attached to, free HyA50 kDa in solution. See FIG. 3.

Example 77—Female BALB/c mice (n=5/cohort) were administered a 5 ug protein equivalent injected 1M as a bolus, followed by a 5 ug IM booster injection at 4 weeks post-prime. Blood was collected via submandibular bleed at 4 and 8 weeks post-prime injection and the serum was analyzed for IgG against the SCH014-RBD-ST3 protein using standard ELISA techniques. Cohorts were immunized with the SCH014-RBD-ST3 protein covalently bound to HyA (HyA25 or HyA50) and the SCH014-RBD-ST3 protein alone. See FIG. 3.

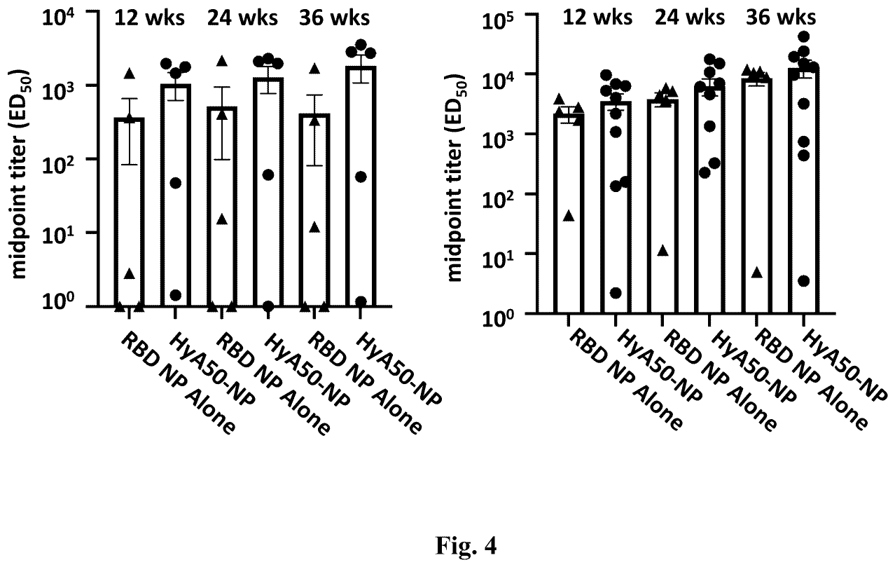

Example 78—BALB/c mice (n=5 to 10/cohort) were administered 1, 10, or 40 ug equivalent dosages of the mosaic-8b RBD-mi3 nanoparticle with and without covalently attached HyA50 via oral gavage twice a week over a 4-week period. Blood was collected via submandibular bleed post-prime delivery and the serum analyzed for serum IgG using standard ELISA techniques. A nasal lavage wash was collected from these same cohorts by administration of 3-4 small drops of sterile saline to a single nostril and extracting the resulting nasopharyngeal wash from the oral cavity using a sterile pipette. Nasal lavage was assayed for sIgA using standard ELISA techniques. See FIG. 4.

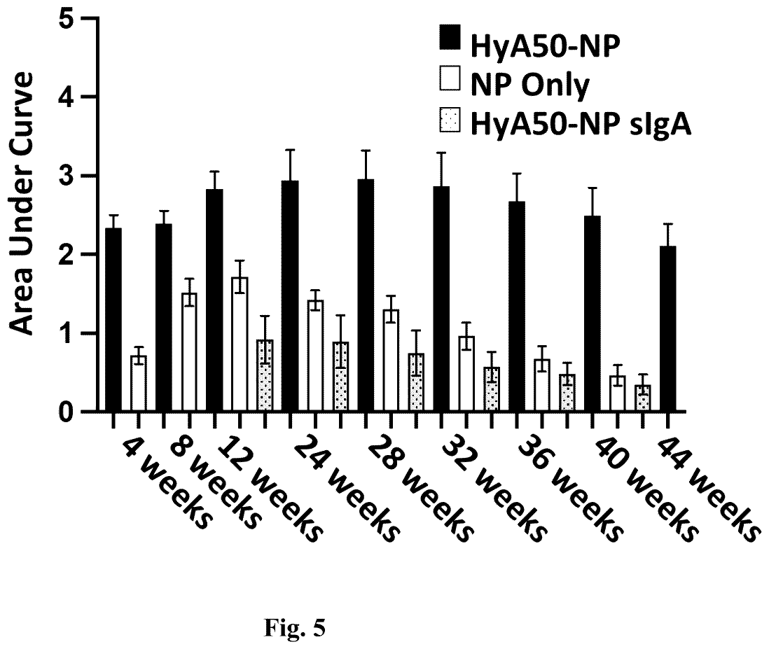

Example 79—BALB/c mice (n=5 to 10/cohort) were administered 0.1, 1, or 10 ug equivalent dosages of the mosaic-8b RBD-mi3 nanoparticle with and without covalently attached HyA50 via 50 ul nasal droplets once per week over a 4-week period. Blood was collected via submandibular bleed post-prime delivery and the serum was analyzed for IgG using standard ELISA techniques. A nasal lavage wash was collected from these same cohorts by administration of 3-4 small drops of sterile saline to a single nostril and extracting the resulting nasopharyngeal wash from the oral cavity using a sterile pipette. Nasal lavage was assayed for sIgA using standard ELISA techniques. See FIG. 5.

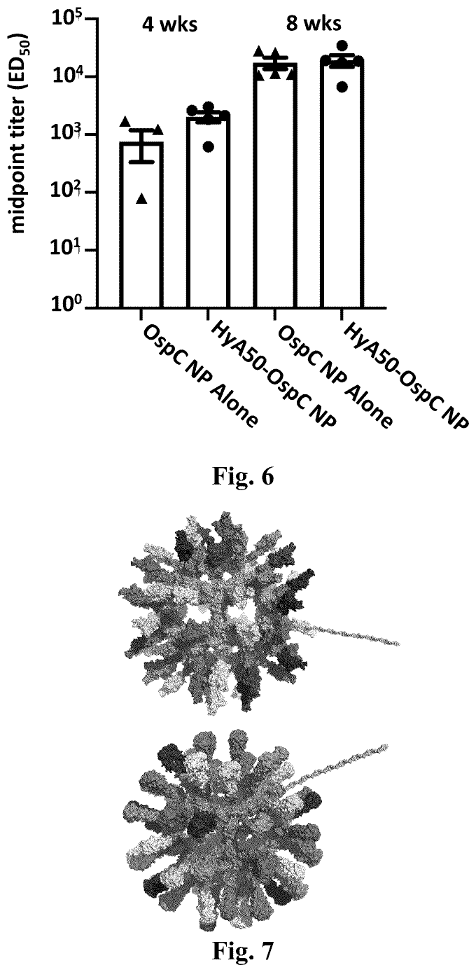

Example 80—Female C3H/HeN mice (n=5 to 10/cohort) were administered a 10 ug protein equivalent of the HyA50-mosaic-8-OspC-mi3 nanoparticle or the mosaic-8-OspC-mi3 nanoparticle IM as a bolus, followed by a 10 ug equivalent IM booster injection at 4 weeks post-prime. Blood was collected via submandibular bleed at 4 and 8 weeks post-prime injection and the serum was analyzed for IgG against the OspC protein using standard ELISA techniques. See FIG. 6.

In order for an antigen to be recognized as a threat in vivo and subsequently trigger antibody production, a recognized series of events must occur (Yin et al., 2021) including maturation of dendritic cells, activation of the MHC-II pathway, surface protein expression of CD80/CD86, expression of OX40L and ICOSL, and activation of T_{FH} and GC responses. FIG. 2 shows that covalent attachment of HyA activates OX40L and ICOSL via the MHCII pathway. This activation is size dependent as different sized HyA polymers showed different activation. Covalent HyA attachment to the mosaic-8b RBD-mi3 nanoparticle induces maturation and licensing of DCs. HyA50 attachment was superior at expression of these proteins as LPS, which is known to be highly antigenic and served as the positive control. These examples establish that covalently attached HyA is capable of the activation of DCs and production of co-stimulatory surface proteins necessary for optimal T_{FH} and GC responses.

The data in FIG. 3-5 establishes that covalently attached HyA is an effective adjuvant in vivo independent of the delivery route (IM, oral, nasal). FIG. 3 demonstrates that covalent attachment of HyA to single protein immunogens acts as an effective adjuvant to stimulate an immune response in the absence of any other traditional adjuvant when delivered by IM injection to BALB/c mice. No other adjuvant is needed to achieve these results.

The results of in vivo experiments with HyA covalently attached to a mosaic-8-RBD nanoparticle demonstrates that HyA attachment does not interfere with development of an immunogenic response as a result of immunization through the mucosal space as shown in FIGS. 4 and 5. The HyA-Mosaic 8-OspC-mi3 nanoparticle can be effectively delivered via mucosal membranes, and demonstration that vaccination using the nanoparticle can provide sufficient protection against a disease challenge. Both oral delivery (FIG. 4) and nasal delivery (FIG. 5) of a nanoparticle are supported by the present disclosure.

Oral cohorts (FIG. 4) exhibited a strong, durable dose-response at each of the timepoints with serum IgG continuing to increase with time. HyA attachment also results in detectable secretory IgA in the nasal cavity following oral administration. Systemic immunity regardless of administration route is predicted by the common mucosal immune system (CMIS) concept wherein lymphocytes induced by an antigen in a mucosal site migrate to other mucosal and systemic sites as effector cells to protect all tissues from the same immunogen.

Nasal cohorts (FIG. 5) exhibited a strong, durable dose-response at each of the timepoints with serum IgG continuing to increase with time. HyA attachment also results in detectable secretory IgA in the nasal cavity following nasal administration. These results further demonstrate the robust ability of HyA, when covalently attached, to elicit effective and lasting production of both serum IgG and secretory IgA in vivo.

Taken together, these results demonstrate the ability of covalently linked HyA to act as a powerful adjuvant when attached to an immunogenic protein or vaccine candidate. Furthermore, these results show that a single length of HyA can act as an effective adjuvant across multiple methods of delivery, including those methods targeting the mucosal membranes, such as intranasal and oral.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth. It will be further understood by those within the art that the meaning of the term “about” would be interpreted to refer to an amount that can range+/−10%. Thus, “at least about” would be interpreted to refer to an amount that can range+/−10% (i.e., at least about a molecular weight of 20,000 Da would be at least 18,000 Da). Conversely, “less than about” would be interpreted to refer to an amount that can range+/−10% (i.e., less than about a molecular weight of 20,000 Da would be less than 22,000 Da).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERENCES

Ahmad, M. et al., Applied Microbiology and Biotechnology 2014, 98 (12), 5301-5317.
Altschul, S. F. et al., J. Mol. Biol. 1990, 215, 403-410.
Ausubel, F. M., Current protocols in molecular biology. Greene Pub. Associates; J. Wiley, order fulfillment: Brooklyn, N.Y. Media, Pa., 2003; p 2 volumes (loose-leaf).
Baker, T. S. et al., Biophys. J. 1991 60, 1445-1456.
Bashiri, S. et al., Pharmaceutics 2020, 12, 965.
Becker, L. C. et al., Int. J. Toxicol. 2009 July-August; 28(4 Suppl):5-67.
Bradford, M. M., Analytical Biochemistry 1976, 72 (1), 248-254.
Brune, K. D. et al., Sci. Rep., 2016, 6, 19234.
Bruun, T. U. et al., ACS Nano, 2018, 12(9), 8855-8866.
Cheung, R. C. et al., Marine drugs 2015, 13 (8), 5156-86.
Cho, Y.-W. et al., Biomacromolecules 2000, 1 (4), 609-614.
Corpet, F. Nuc. Acids Res. 1988, 16(22), 10881-10890.
Cohen, A. A. et al., Science. 2021a, 371(6530):735-741.
Cohen, A. A. et al., PLoS ONE. 2021b, 16(3): e0247963.
Cohen, A. A. et al., Science. 2022, 377 (6606), eabq0839.
Current Protocols in Molecular Biology, Ausubel, F. M. et al. Eds.; John Wiley & Sons, 1994-1998.
D'este, M. et al., U.S. Pat. No. 9,034,624, Issued May 19, 2015.
Earnhart, C. G. et al., Human Vaccines, 2007, 3(6), 281-289.
Ecker, J. W. et al., Vaccines, 2020, 8(3), 462.
Eschenfeldt, W. H. et al., Methods in molecular biology (Clifton, N.J.) 2009, 498, 105-15.
Fearon D. T. Nature, 1997, 388, 323-324.
Gennaro, A. R. (Ed.), Remington's Pharmaceutical Sciences, 18th ed., Mack Publishing Co., Easton, PA, 1990.
Gupta, P. N. Mucosal vaccine delivery and M cell targeting. In Targeted Drug Delivery: Concepts and Design; Devarajan, V. P., Jain, S., Eds.; Springer International Publishing: Cham, Switzerland, 2015; pp. 313-337. ISBN 978-3-319-11354-8.
Hagensee, M. E. et al., J. Virol. 1994, 68, 4503-4505.
Harding, S. E. et al., in Advances in carbohydrate analysis. JAI Press: London, England; Greenwich, Conn., 1991, Vol. 1, pp 63-144.
He, L. et al., Science advances, 2021, 7(12), p.eabf1591.
Hervé, C. et al., NPJ Vaccines, 2019, 4, 39.
Higgins, D. G.; Sharp, P. M. CABIOS 1989, 5(2):151-153.
Higgins, D. G.; Sharp, P. M. Gene, 1988, 73, 237-244.
Hsia, Y.; et al., Nature. 2016, 535(7610), 136-139.
Huang, X.; et al., Bioinfomatics. 1992, 8(2), 155-65,
IUPAC. Compendium of Chemical Terminology, 2nd ed. (the “Gold Book”). Compiled by A. D. McNaught and A. Wilkinson. Blackwell Scientific Publications, Oxford (1997). Online version (2019-) created by S. J. Chalk. ISBN 0-9678550-9-8. https://doi.org/10.1351/goldbook.
Jürgens, D. et al., J. Chromat. A. 1985, 348, 363-370.
Kang, Y. F. et al., ACS nano. 2021, 15(2), 2738-2752.
Keeble, A. H. et al., Proc. Natl. Acad. Sci. USA. 2019, 116 (52), 26523-26533.
Kwang, H. S. et al., Pat. Pub. Appl. No. 20150140149.
Lawson, L. B. et al., Curr. Opin. Immunol. 2011, 23, 414-420.
Li, M. et al., J. Pharm. Investig. 2021, 51, 425-438.
Macpherson, A. J. et al., Mucosal Immunol. 2008, 1, 11.
March, Advanced Organic Chemistry: Reactions, Mechanisms and Structures, 4th Ed., John Wiley & Sons, pp. 69-74 (1992).
Molecular Cloning: a Laboratory Manual, 3^{rd} edition; Russell, D. W.; Sambrook, J., Eds.; Cold Spring Harbor Laboratory Press, 2001.
Møller, H. J.; Poulsen, J. H., Staining of Glycoproteins/Proteoglycans on SDS-Gels. In The Protein Protocols Handbook, Walker, J. M., Ed. Humana Press: Totowa, NJ, 2009; pp 569-574.
Moss, G. P. Pure Appl. Chem. 1996, 68(12), 2193-2222.
Motohashi, N.; Mori, I., Journal of Chromatography A 1984, 299, 508-512.
Müller, P. Pure Appl. Chem. 1994, 66(5), 1077-1184
Needleman, S. B.; Wunsch, C. D. J. Mol. Biol. 1970, 48, 443-453.
Noguchi, M. et al., Helvetica Chimica Acta 2012, 95 (10), 1928-1936.
Nguyen, B.; Tolia, N. H. npj Vaccines, 2021, 6(1), 70.
Obata, A.; Ikushima, K. JP Patent No. H05163161A, Issued Nov. 20, 2000.
O'Hagan, D.; Pavesio, A. U.S. Pat. No. 6,824,793, Issued Nov. 28, 2020.
Olsen, A. W. et al., J. Infect. Dis., 2015, 212(6), 978-989.
Orviský, E. et al., Biomedical Chromatography 1991, 5 (6), 251-255.
Osset, M et al., Electrophoresis 1989, 10 (4), 271-3.
Pearson, W. R.; Lipman, D. J. Proc. Natl. Acad. Sci. USA. 1988, 85, 2444-2448.
Pearson, W. R. Using the FASTA Program to Search Protein and DNA Sequence Databases. In: Computer Analysis of Sequence Data. Methods in Molecular Biology, Griffin, A. M.; Griffin, H. G. Eds. Humana Press, 1994; vol 24, pp. 307-331. https://doi.org/10.1385/0-89603-246-9:307
Petillo, P. A. et al., U.S. Pat. No. 11,021,730, Issued Jun. 1, 2021.
Petillo, P. A. et al., U.S. Pat. No. 11,643,376, Issued Jan. 10, 2023.
Qiagen, The QIAexpressionist™—A handbook for high-level expression and purification of 6×His-tagged proteins. 2003, (5th Edition), 1-126.
Rosato, A et al., U.S. Pat. Appl. Pub. No. 2021/0393758.
Roth, M.; Papakonstantinou, E.; Karakiulakis, G., Chapter 9—Biological Function of Glycosaminoglycans. In Carbohydrate Chemistry, Biology and Medical Applications, Garg, H.
G.; Cowman, M. K.; Hales, C. A., Eds. Elsevier: Oxford, 2008; pp 209-226.
See, Y. P. et al., Electrophoresis 1985, 6 (8), 382-387.
Simon-Loriere, E.; Schwartz, O. Nat. Rev. Microbiol. 2022, 20, 187-188.
Simon, J. et al., U.S. Pat. No. 6,838,086, Issued Jan. 4, 2005.
Smith, T. F. Waterman, M. S. Adv. Appl. Math. 1981, 2, 482-489.
Suzuki, K. et al., Biomater. Sci., 2022, 10, 1920-1928.
Tan, T. K. et al., Nat. Commun. 2021, 12(1), 542.
Yamada, S et al., Communicative & Integrative Biology 2011, 4 (2), 150-158.
Yeung, B.; Marecak, D. J. Chromat. A 1999, 852 (2), 573-581.
Yeung, B. K. S. et al., J. Carbohydr. Chem. 2002, 21, 799-865.
Yin, X. et al., Annu. Rev. Immunol. 2021, 39, 759-790.
Zakeri, B. et al., Proc. Natl. Acad. Sci. USA. 2012; 109 (12), E690-E697.
Zhang, B. et al., Sci. Rep. 2020, 10, 18149.
Zinkernagel, R. M. Immunol Rev. 1997, 156:199-209.
Zonneveld, B. J.; van der Zanden, A. L., Yeast 1995, 11 (9), 823-7.

1. (canceled)

2. (canceled)

3. (canceled)

4. (canceled)

5. (canceled)

6. (canceled)

7. A vaccine composition, comprising a plurality of immunogen molecules attached to a carrier wherein said plurality of immunogen molecules comprises at least one immunogen that has an N-terminal amine and at least one amino nucleophilic moiety, wherein said at least one amino nucleophilic moiety is a lysine and/or said N-terminal amine, and wherein said at least one amino nucleophilic moiety is covalently glycosylated through the reducing end of a linear carbohydrate, said linear carbohydrate functionalized at the reducing end with an oxazoline, said glycosylated immunogen having a molecular weight of at least 7,500 Daltons.

8. (canceled)

9. The vaccine composition of claim 7, wherein said carrier comprises two, three, four, five, six, seven, eight, nine, ten, eleven, or twelve immunogen molecules, each of which is different from one another.

10. The vaccine composition of claim 9, wherein the ratio of any two different immunogen molecules is from about 1:100 to about 100:1.

11. (canceled)

12. The vaccine composition of claim 7, wherein the immunogen molecules of said plurality of immunogens are derived from pathogens independently and individually selected from the group consisting of influenza viruses, rhinoviruses, human immunodeficiency viruses (HIV), respiratory syncytial virus (RSV), coronaviruses, dengue viruses, hepatitis viruses, West Nile virus, Middle East respiratory syndrome-related coronavirus (MERS-CoV), norovirus, Marburg viruses, Zika virus, orthopoxviruses, Togaviridae, Ebola virus, Borrelia, Babesia, methicillin-resistant Staphylococcus aureus (MRSA), Legionella, Chlamydia, Plasmodia, Streptococcus pneumoniae, Vibrio cholerae, Listeria, Clostridia, Salmonella, Bordetella, Enterococci, Treponemia, Amoeba, Neisseria, Anaplasma, and Giardia.

13. (canceled)

14. The vaccine composition of claim 7, wherein said linear carbohydrate is individually and independently selected from the group consisting of hyaluronic acid, partially deacylated chitin, chitosan, and partially acylated chitosan.

15. The vaccine composition of claim 14, wherein said linear carbohydrate has a molecular weight individually and independently selected from the group consisting of at least 33,000, at least 50,000, and at least 120,000 Daltons.

16. (canceled)

17. The vaccine composition of claim 7, wherein said carrier comprises a nanoparticle selected from the group consisting of lipid-based nanoparticles, polymeric nanoparticles, inorganic nanoparticles, surfactant-based emulsions, nanowires, silica nanoparticles, virus-like particles (VLP), a self-assembling nanoparticle, peptide or protein-based particles, lipid-polymer particles, nano lipoprotein particles, and combinations thereof.

18. (canceled)

19. The vaccine composition of claim 17, wherein said virus-like particle is mutated Ap205 VLP.

20. The vaccine composition of claim 17, wherein said carrier is a self-assembling nanoparticle comprising a plurality of particle-forming proteins.

21. The vaccine composition of claim 20, wherein said self-assembling nanoparticle comprises a plurality of particle-forming proteins of 2-dehydro-3-deoxy-phosphogluconate (KDPG) aldolase or a variant thereof.

22. The vaccine composition of claim 21, wherein said self-assembling nanoparticle is selected from the group consisting of an i301 nanoparticle or a variant thereof, and a mi3 nanoparticle or a variant thereof.

23. (canceled)

24. The vaccine composition of claim 21 wherein said immunogen molecules of said plurality of immunogen molecules are covalently attached to said particle-forming protein of said plurality of particle-forming proteins through a SpyTag/SpyCatcher binding pair.

25. (canceled)

26. A method for treating or preventing an infection in a subject in need thereof, comprising administering to the subject a pharmaceutically effective amount of the vaccine composition of claim 7, wherein said vaccine composition administration is independently and individually selected from the group consisting of enteral, oral, parenteral, topical, intranasal, intravaginal, intrarectal, intraocular, and intravitreal, thereby treating or preventing the infection in the subject.

27. (canceled)

28. (canceled)

29. (canceled)

30. The method of claim 26, wherein administering said vaccine composition induces neutralizing and cross-reactive neutralizing responses against additional immunogens different from said immunogens in said plurality of immunogens.

31. The method of claim 26, wherein said vaccine composition is administered to the subject one or more times.

32. The method of claim 31, wherein administering said vaccine composition comprises administering to said subject a first vaccine composition and administering to said subject a second vaccine composition.

33. (canceled)

34. (canceled)

35. The method of claim 32, wherein said administration of said first vaccine composition and said second vaccine composition are independently and individually selected from the group consisting of enteral, oral, parenteral, topical, intranasal, intravaginal, intrarectal, intraocular, and intravitreal.

36. The method of claim 35, wherein administering to said subject said second vaccine composition occurs about two, three, four, five, six, seven, eight weeks, 10 weeks, 12 weeks, 16 weeks, 20 weeks, 24 weeks, 28 weeks, 6 months, 1 year, 5 years, or 10 years, after administering to said subject said first vaccine composition.

37. A kit, comprising the vaccine composition of claim 7, and instructions for administering said vaccine composition to a subject in need thereof.
